(19) United States
(12) Patent Application Publication
(10) Pub. No.: US 2023/0029157 A1
(43) Pub. Date: Jan. 26, 2023

(54) COMBINATION THERAPY FOR HEAD AND NECK CANCER

(71) Applicant: THE REGENTS OF THE UNIVERSITY OF CALIFORNIA, Oakland, CA (US)

(72) Inventors: J. Silvio GUTKIND, La Jolla, CA (US); Zhiyong WANG, La Jolla, CA (US)

(21) Appl. No.: 17/779,499

(22) PCT Filed: Nov. 23, 2020
(86) PCT No.: PCT/US2020/061872

§ 371 Date: May 24, 2022

Related U.S. Application Data
(60) Provisional application No. 62/940,816, filed on Nov. 26, 2019.

(51) Int. Cl. C07K 16/32; C07K 16/28

(52) U.S. Cl. CPC C07K 16/32; C07K 16/2818; A61K 2039/507

(57) ABSTRACT

Disclosed herein are methods of treating HNSCC that comprises administration of a HER3 blocker and an immune checkpoint modulator. In some embodiments, also disclosed herein is a method of modulating activity level of the PI3K/AKT/mTOR pathway in a HNSCC cell to modulate proliferation or sensitization of the cancer cell to a treatment therapy.

### CROSS-REFERENCE TO RELATED PATENT APPLICATION

This application claims priority under 35 U.S.C. § 119(e) to U.S. Provisional Pat. Application No. 62/940,816, filed Nov. 26, 2019, the content of which is hereby incorporated by reference in its entirety.

### BACKGROUND OF THE DISCLOSURE

Squamous cell carcinomas of head and neck (HNSCC), which arise in the oral cavity, oropharynx, larynx and hypopharynx, are a major public health concern. HNSCC is the sixth most common cancer worldwide, with more than 500,000 new cases each year, of which only 40-50% will survive for 5 years. Over 42,000 new cases of HNSCC are predicted to be diagnosed and 8,300 deaths to occur in 2014 from this disease in the United States alone.

Head and neck squamous cell carcinoma (HNSCC) is a significant worldwide health issue with high mortality and morbidity¹: Every year more than 600,000 new cases of HNSCC are diagnosed worldwide, ranking 6th in incidence². The main risk factors include tobacco and alcohol use, and human papillomavirus (HPV) infection³. In the United States, more than 65,400 new cases of HNSCC were predicted to occur in 2019, resulting in 14,600 deaths¹. The incidence of HNSCC is rising with the increasing incidence of HPV+ oropharyngeal cancer^{4,5}. Despite advances in curative intent therapy over the last 3 decades, long-term toxicity continues to be unacceptable for many patients who are cured while those that develop recurrent disease will inevitably succumb⁶. HNSCC has a poor five-year survival rate at 63%^{7,8}. There is an urgent need to develop new approaches to achieve durable cure and relapse-free survival⁶.

Limited response to new immunotherapies in head and neck cancer: HNSCCs deploy multiple mechanisms to avoid immune recognition and subsequent anti-tumor immune response, including the recruitment of myeloid-derived suppressor cells (MDSCs) and conditioning of the surrounding microenvironment to become highly immune suppressive by expressing cytokines, such as IL6, IL10 and TGFβ, leading to the accumulation of suppressive regulatory T cells (Tregs) and the polarization of macrophages toward an immune suppressive (M2) tumor associated macrophage (TAM) phenotype⁹⁻¹¹. A key emerging mechanism of tumor immunosuppression involves T cell exhaustion, whereby T cell reactivity is impaired due to activation of T cell checkpoints, including PD-1, by its ligand, PD-L1 that is expressed by macrophages and some cancer cells, including HNSCC, restraining T cell activation¹²⁻¹⁴. Indeed, new immune check point blockers (ICB), such as pembrolizumab and nivolumab (anti-PD-1) have recently demonstrated potent anti-tumor activity in a subset of HNSCC patients¹⁵⁻¹⁷. These novel T cell targeted therapeutics can re-activate anti-tumor T cell responses; however, one-year survival and response rates of anti-PD-1 in HNSCC were only 36% and 13%, respectively, in a Phase 3 clinical trial¹⁸. This highlights the urgent need to identify novel therapeutic options to increase the effectiveness of ICB for the >80% of patients that do not respond to anti-PD-1 treatment. This disclosure satisfies this need and provides related advantages as well.

### SUMMARY OF THE DISCLOSURE

A striking finding from the recent deep sequencing of the HNSCC genomic landscape was the remarkable multiplicity and diversity of genetic alterations in this malignancy¹⁹⁻²². This makes the search for cancer-driving molecular events daunting. Nonetheless, the emerging picture from the in-depth analysis of the HNSCC oncogenome is that while the specific molecules altered in each individual tumor may be distinct, they all participate in only a handful of network alterations, including those regulated by the TP53, FAT1, NOTCH1, CASP8, CDKN2A (p16^{INK4A}) genes, and PI3K mutations. Among them, PIK3CA, encoding the PI3Kα catalytic subunit, is the most commonly mutated oncogene in HNSCC (~20%), with a significant enrichment of PIK3CA mutations in HPV+ tumors (25%)^{19,23}. In this regard, Applicant contributed the early discovery that the persistent activation of the PI3K/mTOR signaling circuitry is the most frequent dysregulated signaling pathway in HNSCC (>80% of all HPV- and HPV+ cases²⁴⁻²⁷). Applicant also showed that mTOR inhibitors (mTORi) exert potent antitumor activity in multiple experimental HNSCC model systems²⁷⁻³⁴ and in a recent Phase 2 clinical trial³⁵. Thus, while representing a major HNSCC driver, the overreliance on PI3K/mTOR signaling for tumor growth may in turn expose a cancer vulnerability that can be exploited therapeutically. However, the immunosuppressive effects of mTORi limit the potential benefits of their combination with new immune oncology (IO) agents. This disclosure addresses this need.

Thus, provided herein is a method of treating HNSCC that comprises, or consists essentially of, or yet further consists of administration of a HER3 blocker (e.g., an anti-HER3 antibody, fragment, mimetic or an equivalent thereof, optionally an anti-HER3 inhibitory antibody, further optionally CDX-3379 or a fragment or an equivalent thereof) and an immune checkpoint modulator to a subject in need thereof. Responsiveness to therapy includes one or more of reduction in tumor size, volume or burden, longer time to tumor progression, longer overall survival or reduction in disease progression. The therapy is suitable for animals, mammals and humans. Appropriate modes of administration are provided herein.

In some embodiments, also disclosed herein is a method of modulating activity level of the PI3K/AKT/mTOR pathway in a HNSCC cell or tissue containing such cell to modulate proliferation or sensitization of the cancer cell to a treatment therapy. In one aspect, the method comprises, or consists essentially of, or yet further consists of, treating a patient having been identified as benefiting from the treatment. In another aspect, the method comprises, or alternatively consists essentially of, or yet further consists of, determining whether the subject can benefit a treatment with an anti-human epidermal growth factor receptor 3 (HER3) blocker (e.g., an anti-HER3 antibody, fragment, mimetic or an equivalent thereof, optionally an anti-HER3 inhibitory antibody, further optionally CDX-3379 or a fragment or an equivalent thereof) and the immune checkpoint modulator by performing or having performed a diagnostic assay on a biological sample isolated from the patient to determine if the subject can benefit the treatment with the HER3 blocker and the immune checkpoint modulator, e.g., the anti-human epidermal growth factor receptor 3 (HER3) antibody, fragment, mimetic or the equivalent thereof, and the immune checkpoint modulator; and if the subject can benefit the treatment, and then administering to the subject the HER3 blocker and the immune checkpoint modulator (e.g., the anti-human epidermal growth factor receptor 3 (HER3) antibody, fragment, mimetic or equivalent thereof and the immune checkpoint modulator); or if the subject cannot benefit the treatment, do not administer the treatment or the administration of another therapy to the subject. Responsiveness to therapy includes one or more of reduction in tumor size, volume or burden, longer time to tumor progression, longer overall survival or reduction in disease progression. The therapy is suitable for animals, mammals and humans. Appropriate modes of administration are provided herein.

Kits and compositions to provide such therapies are further provided herein.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: Kinome-wide siRNA screen show that HER3 is among the top 20 kinases whose knockdown decreases HNSCC cell proliferation. siRNA library screen targeting 518 kinase with Cal27 cells was conducted to search for genes that affect proliferation of HNSCC. The figure shows genes whose knockdown decreased cell proliferation (Z score). The dark gray regions represent the top 20 genes (which are listed on the right). The HER3 gene is indicated by an arrow. Western blotting was performed to assess pS6 levels after knockdown of each genes by 3 different siRNAs compared to siRNA control.

FIG. 2: Knock down of HER3 suppresses PI3K/mTOR signaling in PIK3CA wild type HNSCC cells. HER3 was knock down with siRNAS in Cal27 cells, which express wild type PIK3CA, and in cells in which PIK3CA H1047R was expressed ectopically and in Detroit562 cells harboring this mutation endogenously. Western blot analysis shows reduction of p-S6 only in cells expressing wild type PIK3CA.

FIG. 3: PIK3CA confers CDX-3379 resistance. Cal27 cells wild type and expressing PIK3CA H1047R mutations developed tumors in nude mice, and treated with vehicle control or CDX-3379. *** P <0.001 when compared to control mice (n=10 per group).

FIG. 4: Reverse phase protein array (RPPA) data from the HNSCC TCGA revealed a shorter survival of patients exhibiting higher levels of tyrosine phosphorylated HER3. TCGA HNSC cases with available RPPA and overall survival data are subject to this analysis (n=212). Expression of tyrosine phosphorylated ERBB3 with pY1289 was assessed by RPPA and separated by z score±0.5 for overall survival.

FIG. 5: PI3K p85/p110 subunits bind directly to HER3. Immunoprecipitation (IP) by HER3 shows direct binding between HER3 and PI3K subunits, which is decreased by HER3 knock down (si-HER3). In contrast, IP by EGFR detected weak binding to p85/p110. EGFR knockdown (si-EGFR) used as control.

FIGS. 6A- 6B: Waterfall plot of change in tumor burden and p-HER3 expression in a window of opportunity trial in HNSCC. Twelve HNSCC patients received CDX-3379 (1000 mg/kg) at a two-week interval for a total of two doses. (FIG. 6A) Change (%) from baseline in the sum of longest diameters of target lesion(s) for each study patient. Radiographic assessments were performed at a median (range) of 20.5 (15-26) days from first CDX-3379 dose. HPV status is denoted by + and - signs. B. (FIG. 6B) Mean tumor pHER3 (pErbB3) levels for all patients (n=12) before and after CDX-3379 treatment. Data from NCT02473731, Grandis et al., manuscript submitted.

FIGS. 7A - 7F: (FIG. 7A) A collection of syngeneic HNSCC mouse cell lines were generated from 4NQO-induced mouse tongue cancers. (FIG. 7B) 4MOSC cells reflect the genetic alterations of human HNSCC. (FIG. 7C) The histology and characteristics of syngeneic tongue cancer tumors resemble human HNSCC. (FIG. 7D) The syngeneic tumors are highly immune infiltrated. Example of a representative flow cytometry analysis (left) and immunofluorescence analysis (n=4). (FIG. 7E) Abundant intratumoral CD8+ T cells exhibit exhaustion characteristics (e.g., high levels of PD-1, CTLA-4, and TIM3). (FIG. 7F) 4MOSC cells are immunogenic. No tumor growth was observed upon long term observation (>2 months) in mice that were previously immunized with irradiated 4MOSC cells (n=10).

FIGS. 8A - 8B: Effect of αPD1 treatment of 4MOSC1 tumors. 4MOSC 1 cells were implanted in the tongue of C57BL/6 mice (500,000 cells) followed by αPD1 treatment (200 µg murine anti-PD-1 G4 Clone 3x weekly) or control IgG (left). (FIG. 8A) Changes in CD8+ T cell infiltration by FACS analysis (n=4) and (FIG. 8B) in gene expression by NanoString technology in 4MOSC1 tumors in response to αPD1 treatment for 5 days (n=3).

FIGS. 9A-9B: CDX-3379 inhibits Her3-PI3K/mTOR signaling and tumor growth in orthotopic 4MOSC1 HNSCC. C57BL/6 mice were implanted with 1×10⁶ 4MOSC1 cells into the tongue, and when they reached approximately 30 mm³, mice were treated with control or CDX3379 (αHER3). Mice were treated once every 3 days for 2 times. (FIG. 9A) Representative immunohistochemical analysis of pS6 were acquired using histological tissue sections from each treatment group. HNSCC cells in the tongue are rounded by a dotted line. (FIG. 9B) Tumor cell proliferation as judged by BrDU staining.

FIG. 10: Rapid changes in tumor cytokines and chemokines in response to CDX-3379. C57BL/6 mice were implanted with 1×10⁶ 4MOSC1 cells into the tongue, and when they reached approximately 30 mm³, mice were treated with control or CDX-3379 (αHER3) antibodies, as for FIG. 10 (n= 4 per group). Tumor lysates were used for Mouse Cytokine Array/Chemokine Array 44-Plex (MD44) analysis, and reported as Log₂(fold change) with respect to control.

FIG. 11: Effect of HER3 and αPD-1 co-targeting in a syngeneic HNSCC model. One million 4MOSC1 were injected into the tongue of C57BL/6 mice (n=10 /group). One week later, established 4MOSC1 tumors were treated with injection of vehicle control (upper left panel), anti-PD-1 (αPD-1) antibody (upper right panel), CDX-3379 (αHER3, lower left panel), and a combination of both (lower right panel). Tumor volume were monitored in individual mice.

FIGS. 12A-12B: Representative IHC analysis of CD8+ T cells in tissue sections from FIG. 11. The percentage of CD8+ T cells were quantified. Kaplan-Meier estimates of overall survival among all animals randomly assigned to vehicle control (1)), PD-1 antibody (αPD-1, (2)), CDX-3379 (αHER3, (3)), and a combination of both (4)). Statistical significance are indicated illustrates representative IHC analysis of CD8+ T cells in tissue sections from FIG. 11.

### DETAILED DESCRIPTION OF THE DISCLOSURE

Throughout this disclosure, patent and technical publications are identified by a citation or an Arabic numeral, for which the citations are provided immediately preceding the claims. All publications, patents, and patent applications mentioned in this specification are herein incorporated by reference to the same extent as if each individual publication, patent, or patent application was specifically and individually indicated to be incorporated by reference.

A mechanism of tumor immunosuppression involves T cell exhaustion, whereby T cell reactivity is impaired due to the activation of T cell checkpoints, including e.g., PD-1, by its ligand, PD-L1 that is expressed by macrophages and cancer cells, restraining T cell activation. Indeed, new immune check point blockers (ICB), such as pembrolizumab and nivolumab (anti-PD-1) have recently demonstrated potent anti-tumor activity in a subset of HNSCC patients. These T cell targeted therapeutics can re-activate anti-tumor T cell responses; however, one-year survival and response rates of anti-PD-1 in HNSCC were only 36% and 13%, respectively, in a Phase 3 clinical trial. This highlights the need to identify therapeutic options to increase the effectiveness of ICB for the >80% of patients that do not respond to anti-PD-1 treatment.

### Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 µL” means “about 5 µL” and also “5 µL.” Generally, the term “about” includes an amount that would be expected to be within experimental error.

As used herein, the term “comprising” is intended to mean that the methods include the recited steps or elements, but do not exclude others. “Consisting essentially of” shall mean rendering the claims open only for the inclusion of steps or elements, which do not materially affect the basic and novel characteristics of the claimed methods. “Consisting of” shall mean excluding any element or step not specified in the claim. Embodiments defined by each of these transition terms are within the scope of this disclosure.

As used herein, a human epidermal growth factor receptor 3 (HER3) blocker comprises a molecule (e.g., a small molecule or a polypeptide) that impairs or blocks the function of the HER3 protein (also referred to as receptor tyrosine-protein kinase erbB-3). In some instances, the HER3 blocker is an anti-HER3 antibody, an HER3 inhibitory antibody, or a fragment, a derivative, or a mimetic of each thereof. These are known in the art and commercially available.

As used herein, the term “antibody” refers to a protein that binds to other molecules (antigens, e.g., HER3) via heavy and light chain variable domains, V_{H} and V_{L}, respectively. Antibodies include full-length antibodies that include two heavy and two light chain sequences. Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains.

Antibodies of the disclosure include polyclonal and monoclonal antibodies. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Antibodies of the disclosure can belong to any antibody class, IgM, IgG, IgE, IgA, IgD, or subclass. Exemplary subclasses for IgG are IgG₁, IgG₂, IgG₃ and IgG₄.

Antibodies of the disclosure include antibody subsequences and fragments. Exemplary antibody subsequences and fragments include Fab, Fab', F(ab')₂, Fv, Fd, single-chain Fv (scFv), disulfide-linked Fvs (sdFv), light chain variable region V_{L}, heavy chain variable region V_{H}, trispecific (Fab₃), bispecific (Fab₂), diabody ((V_{L}—V_{H})₂ or (V_{H}—V_{L})₂), triabody (trivalent), tetrabody (tetravalent), minibody ((scFv—C_{H})₂), bispecific single-chain Fv (Bis—scFv), IgGdeltaCH2, scFv—Fc, (scFv)₂—Fc and IgG4PE. Such subsequences and fragments can have the binding affinity as the full length antibody, the binding specificity as the full length antibody, or one or more activities or functions of as a full length antibody, e.g., a function or activity of HRF binding antibody.

Antibody subsequences and fragments can be combined. For example, a V_{L} or V_{H} subsequences can be joined by a linker sequence thereby forming a V_{L}-V_{H} chimera. A combination of single-chain Fvs (scFv) subsequences can be joined by a linker sequence thereby forming a scFv — scFv chimera. Antibody subsequences and fragments include single-chain antibodies or variable region(s) alone or in combination with all or a portion of other subsequences.

In some instances, antibody subsequences are also referred to herein as equivalents.

As used herein, the term “equivalent thereof” in reference to a reference protein, polypeptide or nucleic acid, intends those having minimal homology while still maintaining desired structure or functionality. Unless specifically recited herein, it is contemplated that any of the antibodies described herein also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. In some instances, an equivalent in reference to a reference antibody intends at least about 70% homology or identity, or at least 80% homology or identity and alternatively, or at least about 85%, or alternatively at least about 90%, or alternatively at least about 95%, or alternatively at least 98% percent homology or identity in the framework region of the antibody while the complementarity-determining regions (CDRs) of the antibody remains identical to the reference antibody. Alternatively, when referring to polynucleotides, an equivalent thereof is a polynucleotide that hybridizes under stringent conditions to the reference polynucleotide or its complement.

As used herein, the term “CDR” refers to one of the six hypervariable regions within the variable domains of an antibody that mainly contribute to antigen binding. One exemplary used definitions for the six CDRs is provided by Kabat E. A. et al., (1991) Sequences of proteins of immunological interest. NIH Publication 91-3242).

The term “antibody framework” as used herein refers to the part of the variable domain, either VL or VH, which serves as a scaffold for the antigen binding loops (CDRs) of this variable domain, or further includes the CH2 and CH3 constant region in the context of a full-length antibody. In some cases, the term framework encompasses the region of an antibody outside of the CDRs.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code = standard; filter = none; strand = both; cutoff = 60; expect = 10; Matrix = BLOSUM62; Descriptions = 50 sequences; sort by = HIGH SCORE; Databases = non-redundant, GenBank + EMBL + DDBJ + PDB + GenBank CDS translations + SwissProtein + SPupdate + PIR. Details of these programs can be found at the following Internet address: ncbi.nlm.nih.gov/cgi-binBLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6x SSC to about 10x SSC; formamide concentrations of about 0% to about 25%; and wash solutions from about 4x SSC to about 8x SSC. Examples of moderate hybridization conditions include: incubation temperatures of about 40° C. to about 50° C.; buffer concentrations of about 9x SSC to about 2x SSC; formamide concentrations of about 30% to about 50%; and wash solutions of about 5x SSC to about 2x SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1x SSC to about 0.1x SSC; formamide concentrations of about 55 % to about 75%; and wash solutions of about 1x SSC, 0.1x SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

Antibody subsequences and fragments can be prepared by proteolytic hydrolysis of the antibody, for example, by pepsin or papain digestion of whole antibodies. Antibody subsequences and fragments produced by enzymatic cleavage with pepsin provide a 5S fragment denoted F(ab‘)₂. This fragment can be further cleaved using a thiol reducing agent to produce 3.5S Fab’ monovalent fragments. Alternatively, an enzymatic cleavage using pepsin produces two monovalent Fab' fragments and the Fc fragment directly (see, e.g., U.S. Pat. Nos. 4,036,945 and 4,331,647; and Edelman et al., Methods Enymol. 1:422 (1967)). Other methods of cleaving antibodies, such as separation of heavy chains to form monovalent light-heavy chain fragments, further cleavage of fragments, or other enzymatic or chemical may also be used.

As used herein, the PIK3CA protein encoded by the PIK3CA gene comprises the wild-type full length PIK3CA protein and any variants and fragments thereof. In some instances, the PIK3CA protein comprises a sequence set forth in UniProtKB accession no. P42336.2 (SEQ ID NO: 1).

UniProtKB accession no. P42336.2:

MPPRPSSGELWGHILMPPRILVECLLPNGMIVTLECLREATLITIKHELFKEARK YPLHQLLQDESSYIFVSVTQEAEREEFFDETRRLCDLRLFQPFLKVIEPVGNREEKILNREI GFAIGMPVCEFDMVKDPEVQDFRRNILNVCKEAVDLRDLNSPHSRAMYVYPPNVESSP ELPKHIYNKLDKGQIIVVIWVIVSPNNDKQKYTLKINHDCVPEQVIAEAIRKKTRSMLLS SEQLKLCVLEYQGKYILKVCGCDEYFLEKYPLSQYKYIRSCIMLGRMPNLMLMAKESL YSQLPMDCFTMPSYSRRISTATPYMNGETSTKSLWVINSALRIKILCATYVNVNIRDIDKI YVRTGIYHGGEPLCDNVNTQRVPCSNPRWNEWLNYDIYIPDLPRAARLCLSICSVKGRK GAKEEHCPLAWGNINLFDYTDTLVSGKMALNLWPVPHGLEDLLNPIGVTGSNPNKETP CLELEFDWFSSVVKFPDMSVIEEHANWSVSREAGFSYSHAGLSNRLARDNELRENDKE QLKAISTRDPLSEITEQEKDFLWSHRHYCVTIPEILPKLLLSVKWNSRDEVAQMYCLVK DWPPIKPEQAMELLDCNYPDPMVRGFAVRCLEKYLTDDKLSQYLIQLVQVLKYEQYLD NLLVRFLLKKALTNQRIGHFFFWHLKSEMHNKTVSQRFGLLLESYCRACGMYLKHLNR QVEAMEKLINLTDILKQEKKDETQKVQMKFLVEQMRRPDFMDALQGFLSPLNPAHQL GNLRLEECRIMSSAKRPLWLNWENPDIMSELLFQNNEIIFKNGDDLRQDMLTLQIIRIME NIWQNQGLDLRMLPYGCLSIGDCVGLIEVVRNSHTIMQIQCKGGLKGALQFNSHTLHQ WLKDKNKGEIYDAAIDLFTRSCAGYCVATFILGIGDRHNSNIMVKDDGQLFHIDFGHFL DHKKKKFGYKRERVPFVLTQDFLIVISKGAQECTKTREFERFQEMCYKAYLAIRQHANL FINLFSMMLGSGMPELQSFDDIAYIRKTLALDKTEQEALEYFMKQMNDAHHGGWTTK MDWIFHTIKQHALN (SEQ ID NO: 1).

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a PIK3CA protein.

As used herein, an immune checkpoint modulator comprises a modulator to PD-1, PD-L1, PD-L2, CTLA-4, LAG3, B7—H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some instances, the immune checkpoint modulator is a PD-1 inhibitor. In some cases, exemplary PD-1 inhibitors include, but are not limited to, anti-mouse PD-1 antibody Clone J43 (Cat # BE0033-2) from BioXcell, anti-mouse PD-1 antibody Clone RMP1-14 (Cat # BE0146) from BioXcell, mouse anti-PD-1 antibody Clone EH12, Merck’s MK-3475 anti-mouse PD-1 antibody (Keytruda, pembrolizumab, lambrolizumab), AnaptysBio’s anti-PD-1 antibody known as ANB011, antibody MDX-1 106 (ONO-4538), Bristol-Myers Squibb’s human IgG4 monoclonal antibody nivolumab (Opdivo®, BMS-936558, MDX1106), AstraZeneca’s AMP-514 and AMP -224, and Pidilizumab (CT-011) from CureTech Ltd. In some cases, the PD-1 inhibitor is cemiplimab, nivolumab, pembrolizumab, avelumab, durvalumab, or atezolizumab.

The anti-Erb3 monoclonal antibody CDX-3379 is a human monoclonal antibody directed against the human epidermal growth factor receptor ErbB3 (HER3). The antibody binds to a unique epitope on HER3, thereby preventing ErbB3 phosphorylation and both ligand-dependent and ligand independent ErbB3 signaling. The antibody inhibits cellular proliferation and survival of ErbB3-expressing tumor cells. CDX-3379 is made by Celldex therapeutics (see celldex.com/pipeline/cdx-3379.php, last accessed on Nov. 20, 2020). Other anti-Erb3 inhibitory antibodies and fragments thereof (equivalents to CDX-3379) are disclosed in U.S. Pat. Appl. No. 20130273029.

Cancers that are known as head and neck cancers usually begin in the squamous cells that line the mucosal surfaces inside the head and neck. They also can begin in the salivary glands. They are also further characterized by the area of the head or neck where they begin, e.g., the oral cavity, the pharynyx, the larynyx, the paranasal sinuses and nasal cavity and the salivary glands. In some instances, a head and neck cancer described herein is selected from: laryngeal and hypopharyngeal cancer, nasal cavity and paranasal sinus cancer, nasopharyngeal cancer, oral and oropharyngeal cancer, or salivary gland cancer.

In some embodiments, a head and neck cancer described herein is a human papillomavirus (HPV)-positive tumor.

In some instances, the cell being treated and/or a cancer cell obtained from a subject’s head and neck cancer expresses a PIK3CA gene comprising a modification. In some cases, the modification comprises a substitution in the gene that induced an amino acid mutation. In some cases, the amino acid mutation in a PIK3CA protein comprises a mutation at R115, Y343, G363, E542, E545, C971, R975, or H1047, or a combination thereof, wherein the positions correspond to amino acid positions set forth in UniProtKB accession no. P42336.2. In some cases, the amino acid mutation comprises R115L, Y343C, G363A, E542K, E545K, E545K, C971R, R975S, H1047L, or H1047R. In some cases, a cancer cell obtained from the subject’s head and neck cancer expresses a modification in RAS, AKT, PTEN, mTOR, TSC1, TSC2, PIK3CG, PIK3R1, PIK3R5, PIK3AP1, PIK3C2G, or a combination thereof.

In some instances, a head and neck cancer described herein is a metastatic squamous neck cancer with occult primary.

In some cases, a head and neck cancer described herein is a metastatic head and neck cancer.

In some cases, a head and neck cancer described herein is a relapsed or refractory head and neck cancer.

In some embodiments, a head and neck cancer described herein is resistant to a checkpoint inhibitor therapy such as a PD-1 treatment in the absence of the anti-HER3 blocker (e.g., the anti-HER3 antibody, fragment, mimetic or an equivalent thereof, optionally an anti-HER3 inhibitory antibody, further optionally CDX-3379 or a fragment or an equivalent thereof).

As used herein, the terms “treating,” “treatment” and the like mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. In one aspect, the term “treatment” excludes prophylaxis. In terms of oncology, the treatment can be first line, second line, third line, fourth line or fifth line therapy.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject’s condition. A detectable improvement includes a subjective or objective decrease, reduction, inhibition, suppression, limit or control in the occurrence, frequency, severity, progression, or duration of a symptom caused by or associated with a disease or condition, such as one or more adverse symptoms, disorders, illnesses, pathologies, diseases, or complications caused by or associated with the disease or condition, or an improvement in an underlying cause or a consequence of the disease or condition, or a reversal of the disease or condition.

Treatment can therefore result in decreasing, reducing, inhibiting, suppressing, limiting, controlling or preventing a disease or condition, or an associated symptom or consequence, or underlying cause; decreasing, reducing, inhibiting, suppressing, limiting, controlling or preventing a progression or worsening of a disease, condition, symptom or consequence, or underlying cause; or further deterioration or occurrence of one or more additional symptoms of the disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disease or symptom in the subject, such as one or more adverse symptoms, disorders, illnesses, pathologies, diseases, or complications caused by or associated with a disease or condition. Treatment methods affecting one or more underlying causes of the condition, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject’s condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the disease or condition, over a short or long duration of time (hours, days, weeks, months, etc.). Responsiveness to therapy includes one or more of reduction in tumor size, volume or burden, longer time to tumor progression, longer overall survival or reduction in disease progression.

As used herein, the term “effective amount” refers to a dose or concentration of a therapeutic agent (e.g., an anti-HER3 blocker such as an anti-HER3 antibody described herein, an immune checkpoint modulator described herein, a PD-1 inhibitor described herein, or an additional therapeutic agent described herein) that induces a desired pharmacologic and/or physiologic effect. In some instances, the effective amount refers to a dose or concentration of a therapeutic agent that treats or ameliorate a subject’s disease or condition. In some cases, the effective amount refers to a dose or concentration of a therapeutic agent that after administration to a subject leads to a therapeutic benefit or improvement.

As used herein, the terms “individual(s)”, “subject(s)” and “patient(s)” mean any animal or mammal. In some embodiments, the mammal is a human. In some embodiments, the mammal is a non-human, e.g., canine, feline, bovine, murine, rat, simian, equine, hare, rabbit or leporidae. None of the terms require or are limited to situations characterized by the supervision (e.g. constant or intermittent) of a health care worker (e.g. a veterinarian, a doctor, a registered nurse, a nurse practitioner, a physician’s assistant, an orderly or a hospice worker).

In some embodiments, administration of the anti-HER3 blocker, e.g., the anti-HER3 antibody, fragment, mimetic or equivalent thereof, optionally an anti-HER3 inhibitory antibody, further optionally CDX-3379 or a fragment or an equivalent thereof, and the immune checkpoint modulator promotes accumulation of tumor-infiltration lymphocytes.

In some embodiments, administration of the anti-HER3 blocker, e.g., the anti-HER3 antibody, fragment, mimetic or equivalent thereof, optionally an anti-HER3 inhibitory antibody, further optionally CDX-3379 or a fragment or an equivalent thereof, and the immune checkpoint modulator increases CD8+ T cells in the subject.

In some instances, the anti-HER3 blocker, e.g., the anti-HER3 antibody, fragment, mimetic or equivalent thereof, optionally an anti-HER3 inhibitory antibody, further optionally CDX-3379 or a fragment or an equivalent thereof, is formulated for local administration.

In other instances, the anti-HER3 blocker, e.g., the anti-HER3 antibody, fragment, mimetic or equivalent thereof, optionally an anti-HER3 inhibitory antibody, further optionally CDX-3379 or a fragment or an equivalent thereof, is formulated for systemic administration.

In some cases, the immune checkpoint modulator is formulated for local administration.

In some cases, the immune checkpoint modulator is formulated for systemic administration.

In additional cases, the anti-HER3 blocker (e.g., the anti-HER3 antibody, fragment, mimetic or equivalent thereof, optionally an anti-HER3 inhibitory antibody, further optionally CDX-3379 or a fragment or an equivalent thereof), the immune checkpoint modulator, or a combination thereof are formulated for parenteral administration.

In additional cases, the anti-HER3 blocker (e.g., the anti-HER3 antibody, fragment, mimetic or equivalent thereof, optionally an anti-HER3 inhibitory antibody, further optionally CDX-3379 or a fragment or an equivalent thereof),, the immune checkpoint modulator, or a combination thereof are formulated for intravenous, subcutaneous, intramuscular, intranasal, intra-arterial, intra-articular, intradermal, intraosseous infusion, intraperitoneal, or intratechal administration.

In some cases, an additional therapeutic agent disclosed herein comprises a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP- 16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the therapy comprises a first-line therapy. As used herein, “first-line therapy” comprises a primary treatment for a subject with a cancer. In some instances, the cancer is a primary cancer. In other instances, the cancer is a metastatic or recurrent cancer. In some cases, the first-line therapy comprises chemotherapy. In other cases, the first-line treatment comprises radiation therapy. A skilled artisan would readily understand that different first-line treatments may be applicable to different type of cancers.

In some cases, the therapy comprises a second-line therapy, a third-line therapy, a fourth-line therapy, or a fifth-line therapy. As used herein, a second-line therapy encompasses treatments that are utilized after the primary or first-line treatment stops. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprises a salvage therapy.

In some cases, the additional therapeutic agent comprises a palliative therapy.

In some instances, the additional therapy is surgery.

In some embodiments, the anti-HER3 blocker (e.g., the anti-HER3 antibody, fragment, mimetic or equivalent thereof, optionally an anti-HER3 inhibitory antibody, further optionally CDX-3379 or a fragment or an equivalent thereof), the immune checkpoint modulator, and optionally the additional therapeutic agent are administered simultaneously.

In some embodiments, the anti-HER3 blocker (e.g., the anti-HER3 antibody, fragment, mimetic or equivalent thereof, optionally an anti-HER3 inhibitory antibody, further optionally CDX-3379 or a fragment or an equivalent thereof), the immune checkpoint modulator, and optionally the additional therapeutic agent are administered sequentially.

In some embodiments, the anti-HER3 blocker (e.g., the anti-HER3 antibody, fragment, mimetic or equivalent thereof, optionally an anti-HER3 inhibitory antibody, further optionally CDX-3379 or a fragment or an equivalent thereof) is administered to the subject prior to administering the immune checkpoint modulator, and optionally the additional therapeutic agent.

In some embodiments, the anti-HER3 blocker (e.g., the anti-HER3 antibody, fragment, mimetic or equivalent thereof, optionally an anti-HER3 inhibitory antibody, further optionally CDX-3379 or a fragment or an equivalent thereof) is administered to the subject after administering the immune checkpoint modulator, and optionally the additional therapeutic agent.

In some embodiments, the method enhances one or more of: inhibiting metastasis, progression free survival, tumor growth, or overall survival or decreases toxicity.

In some embodiments, the method enhances tumor regression.

In some embodiments, one or more of the methods described herein further comprise a diagnostic step. In some instances, a sample is first obtained from a subject suspected of having a disease or condition described above or for inducing an immune response in the subject. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper’s fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

### Modes for Carrying Out The Disclosure

### Therapeutic Methods

In some embodiments, described herein is a method of modulating a head and neck cancer cell proliferation, comprising: contacting a plurality of cells comprising a head and neck cancer cell and an immune cell with a HER3 blocker and an immune checkpoint modulator, e.g., an anti-HER3 antibody, fragment or equivalent thereof, and an immune checkpoint modulator for a time sufficient to induce decreased activity level of the PI3K/AKT/mTOR pathway in the head and neck cancer cell, whereby the decreased activity level of the PI3K/AKT/mTOR pathway increases susceptibility of the head and neck cancer cell to an activated immune cell, thereby impairing or inhibiting proliferation of the head and neck cancer cell. In some embodiments, the anti-HER3 antibody is a full-length antibody or a fragment thereof. In some embodiments, the anti-HER3 antibody is an inhibitory anti-HER3 antibody. In some embodiments, the anti-HER3 antibody is CDX-3379, or a fragment or an equivalent thereof. In some cases, the cell is a mammalian cell, optionally from a murine, non-human primate, or human. Appropriate modes of administration are provided herein. Responsiveness can be determined by detecting a change or reduction in cell proliferation.

In some embodiments, the immune checkpoint modulator binds to the immune cell to generate the activated immune cell. In some embodiments, the activity level of the PI3K/AKT/mTOR pathway is decreased by at least 1-fold, 2-fold, 3-fold, 4-fold, 5-fold, 6-fold, 7-fold, 8-fold, 9-fold, 10-fold, 20 fold, 30-fold, 50-fold, 100-fold, or more. In some embodiments, the activity level of the PI3K/AKT/mTOR pathway is decreased by at least 10%, 15%, 20%, 25%,30%, 40%, 50%, 60%, 70%, 80%, 90%, 100%, or more. In some embodiments, the immune checkpoint modulator is a PD-1 inhibitor. In some embodiments, the PD-1 inhibitor is cemiplimab, nivolumab, pembrolizumab, avelumab, durvalumab, or atezolizumab. In some embodiments, the head and neck cancer cell expresses a PIK3CA gene comprising a modification. In some embodiments, the modification comprises a substitution in the gene that induced an amino acid mutation. In some embodiments, the amino acid mutation in a PIK3CA protein comprises a mutation at R115, Y343, G363, E542, E545, C971, R975, or H1047, wherein the positions correspond to amino acid positions set forth in UniProtKB accession no. P42336.2. In some embodiments, the amino acid mutation comprises R115L, Y343C, G363A, E542K, E545K, E545K, C971R, R975S, H1047L, or H1047R. In some embodiments, the plurality of cells are located within a tumor microenvironment.

In some embodiments, disclosed herein is a method for selecting a subject for treatment with an anti-human epidermal growth factor receptor 3 (HER3) blocker (e.g., an anti-HER3 antibody, fragment or equivalent thereof, optionally an anti-HER3 inhibitory antibody, further optionally, CDX-3379, or a fragment or an equivalent thereof), and an immune checkpoint modulator, wherein the subject has a head and neck cancer, the method comprising the steps of: determining whether the subject can benefit a treatment with the anti-HER3 blocker (e.g., the anti-HER3 antibody, fragment or equivalent thereof, optionally an anti-HER3 inhibitory antibody, further optionally CDX-3379 or a fragment or an equivalent thereof), and the immune checkpoint modulator by: performing or having performed a diagnostic assay on a biological sample isolated from the subject to determine if the subject can benefit the treatment with the anti-HER3 blocker (e.g., the anti-HER3 antibody, fragment or equivalent thereof, optionally an anti-HER3 inhibitory antibody, further optionally CDX-3379 or a fragment or an equivalent thereof), and the immune checkpoint modulator; and if the subject can benefit the treatment, administering to the subject the anti-HER3 blocker (e.g., the anti-HER3 antibody, fragment or equivalent thereof, optionally an anti-HER3 inhibitory antibody, further optionally CDX-3379 or a fragment or an equivalent thereof), and the immune checkpoint modulator; or if the subject cannot benefit the treatment, do not administer the treatment. In some embodiments, the anti-HER3 antibody is a full-length antibody or a fragment thereof. In some embodiments, the anti-HER3 antibody is an inhibitory anti-HER3 antibody. In some embodiments, the anti-HER3 antibody is CDX-3379, or a fragment or an equivalent thereof. Responsiveness to therapy includes one or more of reduction in tumor size, volume or burden, longer time to tumor progression, longer overall survival or reduction in disease progression. The therapy is suitable for animals, mammals and humans. Appropriate modes of administration are provided herein.

In some embodiments, the subject can benefit the treatment if the subject expresses a level of PD-1 on a plurality of activated T cells that when blocked, is sufficient to activate tumor-killing activity of the plurality of T cells. In some embodiments, the level of PD-1 expression on the plurality of activated T cells is elevated compared to a level of PD-1 expression on a similar number of T cells obtained from a healthy subject. In some embodiments, the level of PD-1 expression is elevated by about 10%, 20%, 30%, 40%, 50%, 60%, 70%, 80%, 90%, 100%, or more. In some embodiments, the level of PD-1 expression is elevated by about 1-fold, 2-fold, 3-fold, 4-fold, 5-fold, 6-fold, 7-fold, 8-fold, 9-fold, 10-fold, 20-fold, 50-fold, or more. In some embodiments, the subject can benefit the treatment if cells of the head and neck cancer express an elevated level of HER3. In some embodiments, the elevated level of HER3 is compared to an expression level of HER3 in a healthy subject. In some embodiments, the expression level of HER3 is elevated by about 10%, 20%, 30%, 40%, 50%, 60%, 70%, 80%, 90%, 100%, or more. In some embodiments, the expression level of HER3 is elevated by about 1-fold, 2-fold, 3-fold, 4-fold, 5-fold, 6-fold, 7-fold, 8-fold, 9-fold, 10-fold, 20-fold, 50-fold, or more. In some embodiments, the subject can benefit the treatment if cells of the head and neck cancer have an elevated activation or activity level in the PI3K/AKT/mTOR pathway. In some embodiments, the elevated activation or activity level in the PI3K/AKT/mTOR pathway is compared to an activation or activity level of the PI3K/AKT/mTOR pathway of cells in a healthy subject. In some embodiments, the activation or activity level in the PI3K/AKT/mTOR pathway is elevated by about 10%, 20%, 30%, 40%, 50%, 60%, 70%, 80%, 90%, 100%, or more. In some embodiments, the activation or activity level in the PI3K/AKT/mTOR pathway is elevated by about 1-fold, 2-fold, 3-fold, 4-fold, 5-fold, 6-fold, 7-fold, 8-fold, 9-fold, 10-fold, 20-fold, 50-fold, or more. In some embodiments, the subject can benefit the treatment if cells of the head and neck cancer express a PIK3CA gene comprising a modification that results in an elevated activation or activity level of the PI3K/AKT/mTOR pathway.

In some embodiments, the modification comprises a substitution in the gene that induced an amino acid mutation. In some embodiments, the amino acid mutation in a PIK3CA protein comprises a mutation at R115, Y343, G363, E542, E545, C971, R975, or H1047, or a combination thereof, wherein the positions correspond to amino acid positions set forth in UniProtKB accession no. P42336.2. In some embodiments, the amino acid mutation comprises R115L, Y343C, G363A, E542K, E545K, E545K, C971R, R975S, H1047L, or H1047R.

In some embodiments, the biological sample is a tumor biopsy sample. In some embodiments, the biological sample is a liquid sample, optionally a blood sample or peripheral blood lymphocytes. In some embodiments, the biological sample is a cell-free DNA sample. In some embodiments, the diagnostic assay is a microarray analysis, quantitative PCR analysis, or RNA sequencing analysis. In some embodiments, the diagnostic assay is an immunofluorescence analysis. In some embodiments, the immune checkpoint modulator is a PD-1 inhibitor. In some embodiments, the PD-1 inhibitor is cemiplimab, nivolumab, pembrolizumab, avelumab, durvalumab, or atezolizumab.

In some embodiments, the head and neck cancer is a human papillomavirus (HPV)-positive tumor. In some embodiments, the head and neck cancer is selected from: laryngeal and hypopharyngeal cancer, nasal cavity and paranasal sinus cancer, nasopharyngeal cancer, oral and oropharyngeal cancer, or salivary gland cancer. In some embodiments, the head and neck cancer is a metastatic squamous neck cancer with occult primary. In some embodiments, the head and neck cancer is a metastatic head and neck cancer. In some embodiments, the head and neck cancer is a relapsed or refractory head and neck cancer. In some embodiments, the head and neck cancer is resistant to a PD-1 treatment in the absence of the anti-HER3 antibody, fragment or equivalent thereof.

In some embodiments, the methods described herein encompass an in vivo method.

In some embodiments, the methods described herein encompass an in vitro or ex vivo method, e.g., in selecting a subject who would benefit from the therapy. Exemplary assays include microarray analysis, quantitative PCR analysis, RNA sequencing analysis, or immunofluorescence analysis.

In some embodiments, the anti-HER3 blocker is an anti-HER3 antibody, fragment or equivalent thereof. In some embodiments, the anti-HER3 antibody is a monoclonal antibody, fragment or equivalent thereof and of any species as it appropriate, e.g., murine or human for example. In some embodiments, the anti-HER3 antibody is a full-length antibody or a fragment thereof. In some embodiments, the anti-HER3 antibody is an inhibitory anti-HER3 antibody. In some embodiments, the anti-HER3 antibody is CDX-3379, or a fragment or an equivalent thereof. In some embodiments, the anti-HER3 antibody, fragment or equivalent thereof is formulated for local administration. In some embodiments, the anti-HER3 antibody, fragment or equivalent thereof is formulated for systemic administration.

In some embodiments, the immune checkpoint modulator is formulated for local administration. In some embodiments, the immune checkpoint modulator is formulated for systemic administration.

In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof), the immune checkpoint modulator, or a combination thereof are formulated for parenteral administration. In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof), the immune checkpoint modulator, or a combination thereof are formulated for intravenous, subcutaneous, intramuscular, intranasal, intra-arterial, intra-articular, intradermal, intraosseous infusion, intraperitoneal, or intratechal administration.

In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof), and the immune checkpoint modulator is formulated as a single dosage form. In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof), and the immune checkpoint modulator is formulated as separate dosage forms.

In some embodiments, the method comprises administering of the anti-HER3 blocker (e.g., the anti-HER3 antibody, fragment or equivalent thereof, optionally an anti-HER3 inhibitory antibody, further optionally CDX-3379 or a fragment or an equivalent thereof), the immune checkpoint modulator, and an additional therapeutic agent or an additional therapy to the subject. In some embodiments, the additional therapeutic agent comprises chemotherapeutic agent, an immunotherapeutic agent, a targeted therapy, radiation therapy, or a combination thereof. In some embodiments, the additional therapeutic agent comprises a first-line therapy. In some embodiments, the additional therapeutic agent comprises a second-line therapy, a third-line therapy, a fourth-line therapy, or a fifth-line therapy. In some embodiments, the additional therapy is surgery.

In some instances, the additional therapeutic agent or an additional therapy is administered to the subject prior to administering the anti-HER3 blocker (e.g., the anti-HER3 antibody, fragment or equivalent thereof, optionally an anti-HER3 inhibitory antibody, further optionally CDX-3379 or a fragment or an equivalent thereof) and the immune checkpoint modulator. In some instances, the additional therapeutic agent or an additional therapy is administered to the subject prior to administering the anti-HER3 blocker (e.g., the anti-HER3 antibody, fragment or equivalent thereof, optionally an anti-HER3 inhibitory antibody, further optionally CDX-3379 or a fragment or an equivalent thereof) and the immune checkpoint modulator is administered last. In other instances, the additional therapeutic agent or an additional therapy is administered to the subject prior to administering the immune checkpoint modulator and the anti-HER3 blocker (e.g., the anti-HER3 antibody, fragment or equivalent thereof, optionally an anti-HER3 inhibitory antibody, further optionally CDX-3379 or a fragment or an equivalent thereof) is administered last. In additional instances, the additional therapeutic agent or an additional therapy is administered to the subject prior to administering the anti-HER3 blocker (e.g., the anti-HER3 antibody, fragment or equivalent thereof, optionally an anti-HER3 inhibitory antibody, further optionally CDX-3379 or a fragment or an equivalent thereof) and the immune checkpoint modulator, in which the anti-HER3 blocker (e.g., the anti-HER3 antibody, fragment or equivalent thereof, optionally an anti-HER3 inhibitory antibody, further optionally CDX-3379 or a fragment or an equivalent thereof) and the immune checkpoint modulator are administered simultaneously.

In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof) and the immune checkpoint modulator (either simultaneously or sequentially) are administered prior to administering the additional therapeutic agent or the additional therapy. In one instances, the anti-HER3 blocker (e.g., the anti-HER3 antibody, fragment or equivalent thereof, optionally an anti-HER3 inhibitory antibody, further optionally CDX-3379 or a fragment or an equivalent thereof) and the immune checkpoint modulator are administered simultaneously. In another instances, the anti-HER3 blocker (e.g., the anti-HER3 antibody, fragment or equivalent thereof, optionally an anti-HER3 inhibitory antibody, further optionally CDX-3379 or a fragment or an equivalent thereof) and the immune checkpoint modulator are administered sequentially.

In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof), the immune checkpoint modulator, and optionally the additional therapeutic agent are administered simultaneously. In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof, the immune checkpoint modulator, and optionally the additional therapeutic agent are administered sequentially. In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof is administered to the subject prior to administering the immune checkpoint modulator, and optionally the additional therapeutic agent. In some embodiments, the anti-HER3 blocker (e.g., the anti-HER3 antibody, fragment or equivalent thereof, optionally an anti-HER3 inhibitory antibody, further optionally CDX-3379 or a fragment or an equivalent thereof is administered to the subject after administering the immune checkpoint modulator, and optionally the additional therapeutic agent. In some embodiments, the subject is a mammal or a human.

In some embodiments, the pharmaceutical composition and formulations described herein are administered to a subject by multiple administration routes, including but not limited to, parenteral, oral, sublingual, or transdermal administration routes. In some cases, parenteral administration comprises intravenous, subcutaneous, intramuscular, intranasal, intra-arterial, intra-articular, intradermal, intraosseous infusion, intraperitoneal, or intratechal administration. In some instances, the pharmaceutical composition is formulated for local administration. In other instances, the pharmaceutical composition is formulated for systemic administration.

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient’s status does improve, upon the doctor’s discretion the administration of the composition is given continuously, alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient’s conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Pharmaceutical Formulations

In some embodiments, described herein is a pharmaceutical formulation for use in treating a head and neck cancer in a subject in need thereof, comprising: an anti-human epidermal growth factor receptor 3 (HER3) antibody, fragment or equivalent thereof (e.g., CDX-3379, or a fragment or an equivalent thereof); an immune checkpoint modulator; and a pharmaceutically acceptable carrier. In some embodiments, the immune checkpoint modulator is a PD-1 inhibitor. In some embodiments, the PD-1 inhibitor is cemiplimab, nivolumab, pembrolizumab, avelumab, durvalumab, or atezolizumab. In some embodiments, a cancer cell of the subject’s head and neck cancer expresses a PIK3CA gene comprising a modification. In some embodiments, the modification comprises a substitution in the gene that induced an amino acid mutation. In some embodiments, the amino acid mutation in a PIK3CA protein comprises a mutation at R115, Y343, G363, E542, E545, C971, R975, or H1047, or a combination thereof, wherein the positions correspond to amino acid positions set forth in UniProtKB accession no. P42336.2. In some embodiments, the amino acid mutation comprises R115L, Y343C, G363A, E542K, E545K, E545K, C971R, R975S, H1047L, or H1047R. In some embodiments, a cancer cell obtained from the subject’s head and neck cancer expresses a modification in RAS, AKT, PTEN, mTOR, TSC1, TSC2, PIK3CG, PIK3R1, PIK3R5, PIK3AP1, PIK3C2G, or a combination thereof.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington’s Pharmaceutical Sciences, Mack Publishing Co., Easton, Pennsylvania 1975, Liberman, H.A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkinsl999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel^{®}, dibasic calcium phosphate, dicalcium phosphate dihydrate, tricalcium phosphate, calcium phosphate, anhydrous lactose, spray-dried lactose, pregelatinized starch, compressible sugar, such as Di-Pac^{®} (Amstar), mannitol, hydroxypropylmethylcellulose, hydroxypropylmethylcellulose acetate stearate, sucrose-based diluents, confectioner’s sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel^{®}, or sodium starch glycolate such as Promogel^{®} or Explotab^{®}, a cellulose such as a wood product, methylcrystalline cellulose, e.g., Avicel^{®}, Avicel^{®} PH101, Avicel^{®}PH102, Avicel^{®} PH105, Elcema^{®} P100, Emcocel^{®}, Vivacel^{®}, Ming Tia^{®}, and Solka-Floc^{®}, methylcellulose, croscarmellose, or a cross-linked cellulose, such as cross-linked sodium carboxymethylcellulose (Ac-Di-Sol^{®}), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum^{®} HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex^{®}), higher fatty acids and their alkali-metal and alkaline earth metal salts, such as aluminum, calcium, magnesium, zinc, stearic acid, sodium stearates, glycerol, talc, waxes, Stearowet^{®}, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil^{®}, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorb ate-20 or Tween® 20, or trometamol. [0338] Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic^{®} (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Kits

As used herein, a kit or article of manufacture described herein include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

The following examples are intended to illustrate and not limit the inventions as disclosed herein.

### Example 1

In search for alternative approaches, Applicant discovered that persistent HER3 tyrosine phosphorylation underlies PI3K/mTOR activation in most HNSCC cases that do not harbor PIK3CA mutations. Furthermore, it was observed that HER3 inactivating antibodies or anti-HER3 antibodies (CDX-3379) exert potent antitumor activity in PIK3CA wild type (wt) HNSCC models, and encouraging results were obtained with this agent in clinical trials in HNSCC (^{36,37} and see below, FIG. 6). Applicant has now obtained evidence that HER3 inhibition with CDX-3379 reverses the immune suppressive tumor microenvironment, and that HER3 may represent a suitable target for combination therapies with anti-PD-1 blocking antibodies. These results show that targeting HER3 in HNSCC will disrupt the PI3K/mTOR oncogenic signaling network and its initiated immune evasive mechanisms, and that HER3 inactivating antibodies will increase the response to anti-PD-1 treatment as part of a novel rational therapeutic strategy. Ultimately, co-targeting the HER3 signaling circuitry combined with PD-1 blockade represents a novel multimodal precision therapeutic approach for HNSCC aimed at achieving durable responses and cancer remission.

Persistent tyrosine phosphorylation of HER3 underlies aberrant PI3K/mTOR signaling in HNSCC harboring wild type PIK3CA

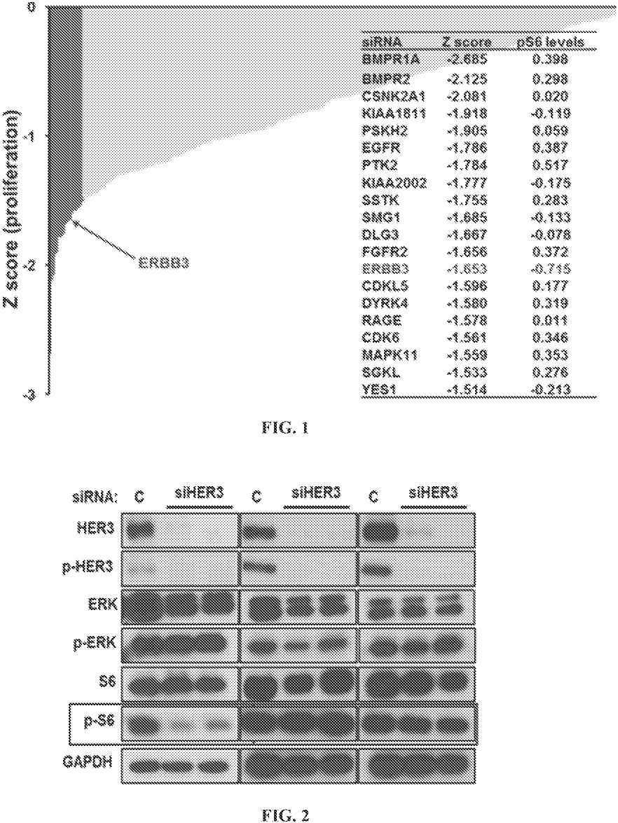

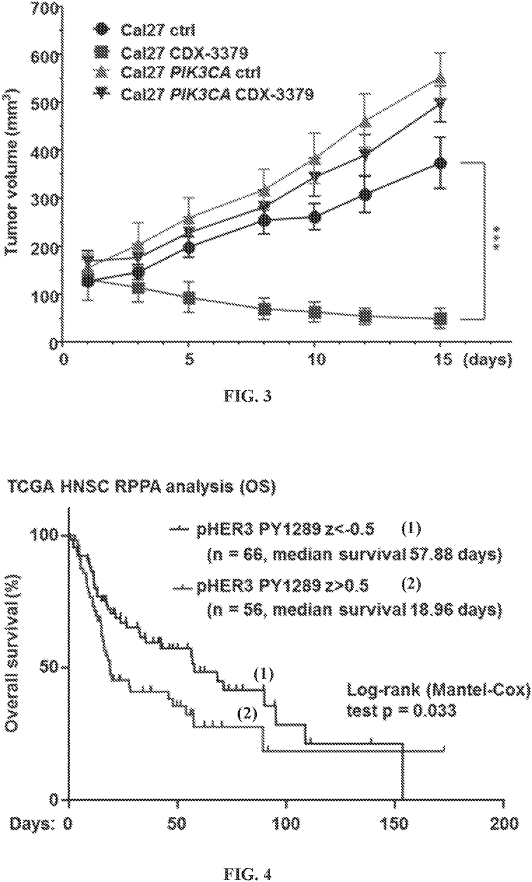

In search for mechanisms controlling elevated PI3K-mTOR activity in HNSCC cells that do not exhibit PIK3CA mutations, Applicant initially conducted a kinome-wide siRNA screen (FIG. 1). The ERBB3 gene, encoding HER3, was among the top 20 kinases whose knockdown (KD) decreased HNSCC cell proliferation. Furthermore, a counter screen analysis of these top hits revealed that ERBB3 was the gene whose KD results in the highest reduction in the phosphorylated form of ribosomal protein S6 (pS6), a downstream target of mTOR used to monitor mTOR pathway activation. Remarkably, HER3 knock down specifically reduced activation of mTOR (as judged by pS6 levels) but not ERK (pERK) in cells that do not harbor PIK3CA mutations, and ectopic or endogenous expression of PIK3CA mutants reverted this activity (FIG. 2). These findings were confirmed in multiple cell lines, and in contrast, EGFR knock down reduced ERK but not mTOR activity (not shown). Similarly, a blocking inhibitory antibody targeting HER3 (CDX-3379) potently inhibited the growth of multiple HPV- and HPV+ PIK3CA wild type HNSCC cells, as previously reported³⁸⁻⁴⁰, but not in PIK3CA mutant HNSCC cells in vitro (not shown) and in vivo (FIG. 3). As a typical HPV- HSNCC cellular system, Cal27 cells tumors are very sensitive to CDX-3379 ³⁹. However, Cal27 tumors become resistant after expression of PIK3CA mutant, similar to Detroit562 (PIK3CA+ HNSCC cells (not shown), supporting that PI3K-mTOR (see below) may represent a key target downstream from HER3 .

HER3 as a therapeutic target in HNSCC; distinguishing properties of CDX-3379

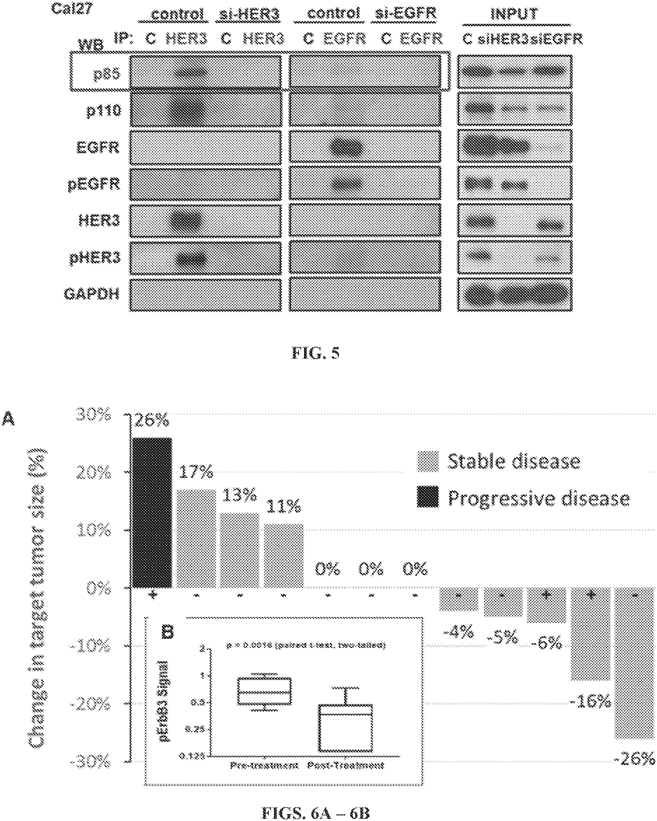

HER3 has been studied extensively as a rapidly activated compensatory pathway promoting resistance to anti-EGFR therapies⁴¹⁻⁴⁴. HER3 is a kinase-deficient receptor that is phosphorylated in a ligand (neuregulin-NRG/heregulin-HRG)-dependent manner through the formation of obligate heterodimers with other HER receptors (EGFR and HER2, both widely expressed in HNSCC^{39,45,46}. Several studies indicated that HNSCC express the highest levels of the HER3 ligand NRG among all cancer types, correlating with poor prognosis^{39,47}, thereby activating HER3 in an autocrine fashion. Aligned with these observations, Applicant’s analysis of tyrosine phosphorylated HER3 (pHER3-PY1289) in the HNSCC TCGA dataset demonstrated a significantly lower overall survival (OS) in patients harboring high levels of pHER3 (FIG. 4). Unlike EGFR and HER2, the intracellular domain of HER3 contains six consensus tyrosine motifs that upon phosphorylation provide high affinity binding to the p85 regulatory subunit of PI3K⁴⁸, thereby eliciting very potent activation of the PI3K/AKT/mTOR signaling axis^{45,49,50}. As such, HER3 may represent a key signaling hub in HNSCC. Indeed, Applicant found that in HNSCC cells a large fraction of PI3K protein is persistently associated with HER3 (but not EGFR) under basal conditions (FIG. 5). In addition, elegant prior studies documented the preclinical activity of targeting HER3 alone and enhanced activity when co-targeting EGFR in multiple HNSCC preclinical models^{39,42,44,51}. However, translation of these findings to the clinic has been challenging. Two HER3 targeting antibodies, duligotuzumab (an EGFR/HER3 bispecifc mAb) and patritumab (an ErbB3-targeting mAb, dosed in combination with cetuximaFb) failed to demonstrate benefit over cetuximab alone in respective phase 2 trials^{52,53}. While initially discouraging, review of clinical and preclinical data suggests that these agents may not have been sufficiently potent to effectively compete with the high affinity ligand NRG, and/or dosed at sufficiently high levels to achieve meaningful target inhibition^{52,54}. By contrast, CDX-3379 has been selected and engineered to overcome these limitations. The crystal structure of CDX-3379 binding to HER3 has been solved, which revealed that it binds HER3 outside of the NRG-ligand binding domain and locks HER3 in its auto-inhibited configuration, making the HER3 incapable of binding ligand or dimerizing with other receptors^{55,56}. Current data indicate that CDX-3379 inhibits ErbB3 via two distinct primary mechanisms of action, ligand-dependent (i.e. NRG-driven HER3 activation) and ligand-independent (i.e. activation driven by high activity of EGFR, HER2, or other tyrosine kinases). Indeed, CDX-3379 binds HER3 with a KD of 100 pM, and inhibits NRG-dependent proliferation of tumor cells significantly more potently than patritumab or seribantumab, a first-in-class HER3-targeting mAbs (Celldex, unpublished observations). The Fc portion of CDX-3379 was engineered to enhance binding to the neonatal Fc receptor (FcRN) and thus enhancing its serum half-life in patients and target exposure. These advantages have been borne out in the clinic; CDX-3379 has a calculated serum half-life of 17 days, which combined with its potency ensures inhibition of HER3 phosphorylation in HNSCC tumors from patients, as demonstrated in a recently completed window-of-opportunity study (FIG. 6). Moreover, CDX-3379 has demonstrated clinical activity as a monotherapy (NCT02014909)³⁶ and in combination with cetuximab (NCT03254927)³⁷ in this setting. Lastly, CDX-3379 potently cross-reacts with and inhibits murine HER3, thus enabling monitoring for the first time the impact of HER3 inhibition on the TIME, and the potential benefits of combining CDX-3379 with ICB in newly developed mouse preclinical HNSCC models. Indeed, Applicant’s studies support that HER3 inhibition reverses the immune suppressive TIME, and that HER3 may represent a suitable target for combination therapies with anti-PD-1 ICB.

Novel syngeneic HNSCC animal models

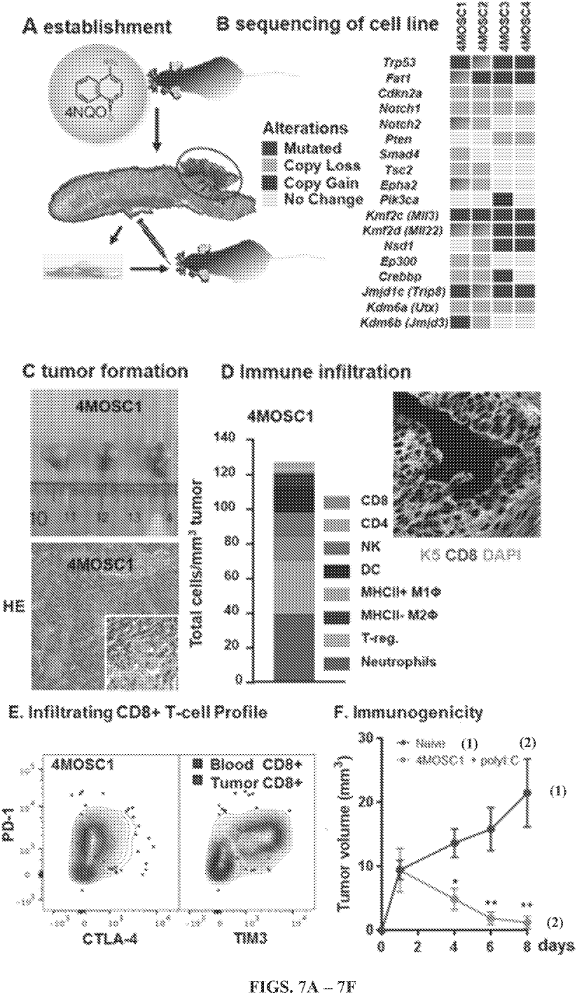

Animal models with a full functioning immune system are critically needed to more accurately recapitulate the complexity of the tumor microenvironment. Dr. Lee developed syngeneic HPV+ mouse model, designated MEER⁵⁷, and Dr. Uppaluri established a panel of mouse HNSCC cell lines from DMBA-treated mice (including MOC1 and MOC2)58. These model systems are driven by Ras oncogenes, and develop tumors in the flank of immune competent C57BL/6 mice^{57,58}. Applicant successfully optimized a carcinogen-induced oral cancer mouse model in which the compound 4-nitroquinoline-1 oxide (4NQO), a DNA adduct-forming agent causing DNA damage mimicking that induced by cigarette smoke, promotes oral cancer initiation²⁹, which has been used extensively to study HNSCC progression⁵⁹⁻⁶⁵. Applicant also has recently developed the first murine HNSCC cell line collection from this relevant carcinogen-induced tongue cancer mouse model (FIG. 7). These cells exhibit typical HNSCC histology and mutations and copy number variations (loss or gains) impacting on Trp53, Fat1, Cdkn2a, Notch2, and M112 and M113 which represent frequently altered gene pathways in HPV-human HNSCC⁶⁶. Remarkably, the mutational signature (mutanome) of these cells is 94% identical to tobacco-related HNSCC⁶⁷ (Pearson correlation > 0.93, manuscript in preparation). 4MOSC1 and 4MOSC2 have been characterized extensively, as they exhibit distinct cancer-immune environments and consequent response to IO agents (see below), reflecting nicely the most typical situations in the clinic. 4MOSC1 tumors are well differentiated, and by FACS analysis it has been shown that they exhibit abundant immune infiltration including cytotoxic and helper T cells, T-regs, NK cells, macrophages, myeloid derived cells, and B cells. Intratumoral CD8+ T cells exhibit exhausted characteristics, including high levels of PD-1, CTLA-4, and remarkably high levels of TIM3 when compared to circulating T cells (FIG. 7). 4MOSC1 tumors express PD-L1 in approximately 9% of SCC cells. 4MOSC cells are immunogenic, as reflected by the fact that vaccination with irradiated cells promote the rejection of a subsequent inoculation of these cells (FIG. 7). 4MOSC2 tumors are less differentiated, do not express PD-L1 (<1% of cells), and exhibit abundant immune infiltration, the majority of which exhibit typical MDSC markers (not shown).

Similarly to the clinical setting, ~20% of 4MOSC1 tumors respond to anti-PD1 treatment, while most mice succumb to the disease burden (See below). Applicant has confirmed that the response to PD-1 blockade is dependent on CD8+ T cells as it is abolished by antibody-dependent T cell depletion (not shown). By FACS, Applicant observed an increase in CD8+ TILs, while NanoString analysis of RNA from SCC lesions of mice treated with anti-PD-1 revealed a remarkable increase in cytotoxic T cells (FIG. 8). Approximately 20% 4MOSC1 tested so far respond to anti-PD1 as a single agent in multiple independent experiments (n=28), suggesting that 4MOSC1 are representative of PD-L1+ HNSCC lesions (~20% of which respond to αPD-1)¹⁵. In contrast, no responses to anti-PD-1 were observed in 4MOSC2 cells (not shown). This new collection of tumorigenic syngeneic mouse HNSCC cell lines reflecting the mutational landscape of human HNSCC and the team’s expertise in anti-tumor immune response analysis will be deployed for the planned studies described in this proposal.

CDX-3379 inhibits HER3 signaling to PI3K/mTOR in syngeneic HNSCC mouse models, and remodels the TIME, enhancing CD8+ T cell recruitment

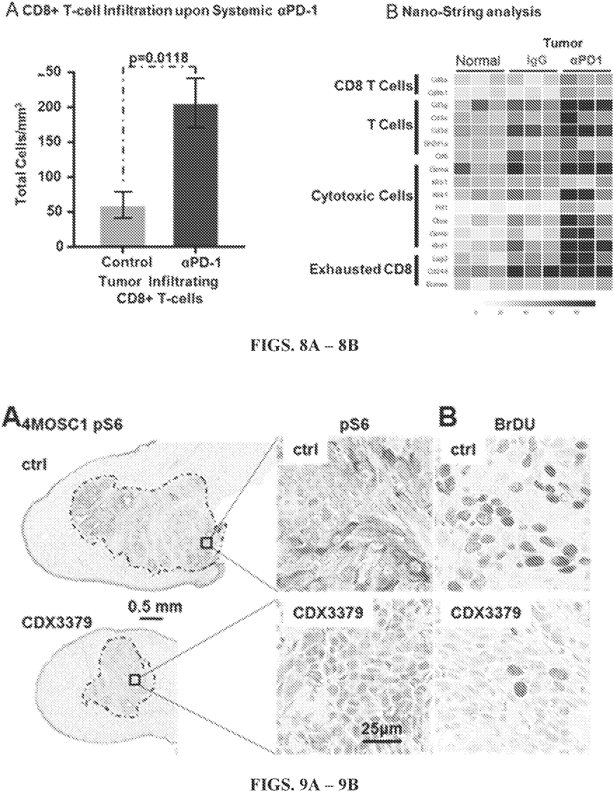

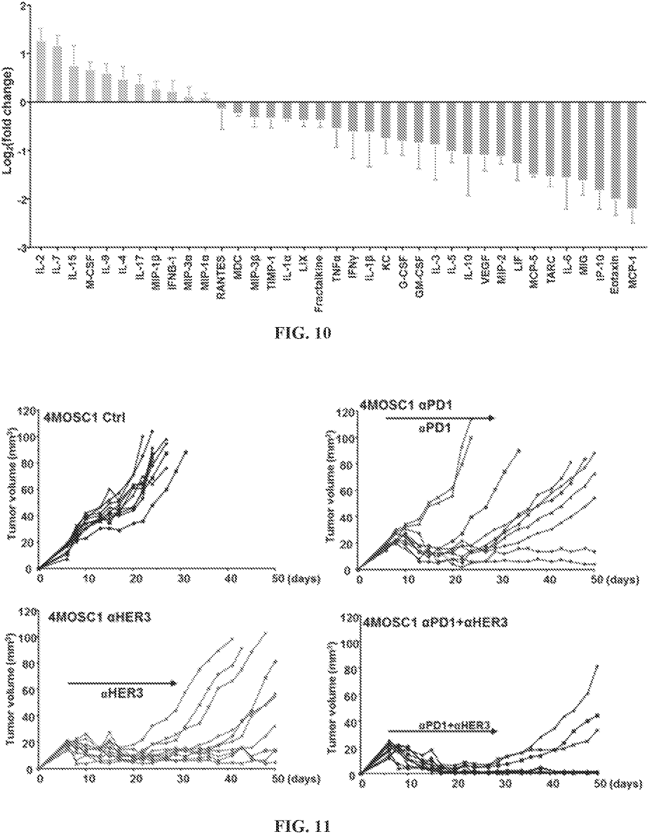

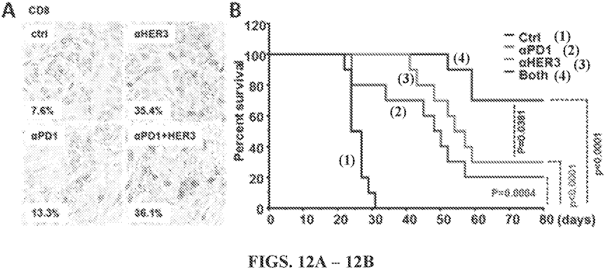

The availability of the first HER3 blocking antibody that recognizes both human and mouse HER3 enabled Applicant to investigate the direct impact of blocking HER3-PI3K/mTOR signaling in cancer cells on their TIME. For this Applicant initially conducted a short term treatment (5 days) of mice bearing tongue 4MOSC1 tumors with CDX-3379 (FIG. 9). Of direct relevance to the planned project, CDX-3379 decreased tumor pS6 staining (surrogate for mTOR activity), and the proliferation of tumor cells (as judged by BrDU staining), supporting the blockade of a functional HER3-PI3K/mTOR growth promoting pathway in this syngeneic tumors (FIG. 9). Applicant observed a rapid decrease in the tumor volume caused by HER3i and increased intratumoral CD8+ T cell infiltration (see below, FIGS. 11 and 12). Direct measurements of cytokine/chemokine levels in tumors revealed that CDX-3379 treatment led a rapid increase in the accumulation of pro-immunogenic IL-2 and IL-7, concomitant with a reduction in multiple pro-tumorigenic and immune suppressive cytokines (e.g., IL-10, VEGF, G-CSF, GM- CSF, IL-6) and chemokines (e.g., MCP-⅟CCL2, KC/CXCL1), albeit HER3i also reduced some pro-immunogenic chemokines (IP-10/CXCL10, MIG/CXCL9) consistent with a decrease in IFN-y (FIG. 10). Surprisingly, Applicant also observed that CD8 T cell depletion reduces the response to CDX-3379 in immune competent mice (day 30 of treatment, IgG control+CDX-3379: 17.38 ± 3.55 mm³, n=10, similar to FIG. 11; anti-CD8+CDX-3379: 63.19 ± 8.40 mm³, n=5, p<0.01), thus raising the possibility that anti-tumor immune responses may contribute to the efficacy of HER3i in HNSCC.

HER3 inactivation increases the response to anti-PD-1 blockade in syngeneic orthotopic mouse HNSCC models

In long term studies, Applicant found that CDX-3379 causes rapid tumor growth inhibition, being as potent as anti-PD-1 both in terms of tumor growth delay (FIG. 11) and CD8+ T cell tumor infiltration and overall survival (FIG. 12). However, as for nearly all targeted therapies, most HER3i treated tumors relapse after treatment termination (FIG. 11), or on treatment (not shown). In this model, in which mouse HNSCC cells are grown orthotopically in the tongue of immune competent C57BL/6 mice, many of the tumors initially respond to anti-PD-1 blockade, but then start to develop resistance over the next few weeks, with only 10-20% of all tumors responding to anti-PD-1 blockade (FIG. 11), leading to durable responses (no regrowth upon prolonged observation of >6 months). This model offers a great opportunity to identify treatments that could diminish resistance by sensitizing the tumors toanti-PD-1 blockade, which could either prolong the phase of stable disease or enhance tumor regression. Indeed, the combination of HER3 and PD-1 blockade elicited a remarkable beneficial effect, with 70% of the mice exhibiting complete and durable (>6 months) responses and consequently significantly increased survival (FIG. 12). Applicant confirmed these findings using a different syngeneic mouse HNSCC model, MOC1 cells, which also responded to the HER3/anti-PD-1 combination: 80% mice responded to anti-PD1 but among responders 50% regrew after treatment termination, while HER3 alone caused a growth delayed, but when combined with anti-PD1 80% of the mice achieved durable remission. These findings increased the rigor of the observations, and together with additional preliminary results provide the basis for the application.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### EMBODIMENTS

Embodiment 1: A method for treating a head and neck cancer in a subject in need thereof, comprising administering to the subject an anti-human epidermal growth factor receptor 3 (HER3) blocker, optionally an anti-HER3 antibody, fragment or equivalent thereof, and an immune checkpoint modulator. In one embodiment, the anti-HER3 antibody is an inhibitory antibody. Non-limiting examples of such include the CDX-3379 antibody, fragments and equivalents thereof.

Embodiment 2: The method of embodiment 1, wherein the anti-HER3 blocker, optionally the anti-HER3 antibody, fragment or equivalent thereof, modulates activation and/or activity of the PI3K/AKT/mTOR pathway.

Embodiment 3: The method of embodiment 1 or 2, wherein the anti-HER3 blocker, optionally the anti-HER3 antibody, fragment or equivalent thereof, decreases PI3K/AKT/mTOR activity.

Embodiment 4: The method of embodiment 2 or 3, wherein the activation and/or activity of the PI3K/AKT/mTOR pathway is decreased by at least 1-fold, 2-fold, 3-fold, 4-fold, 5-fold, 6-fold, 7-fold, 8-fold, 9-fold, 10-fold, 20 fold, 30-fold, 50-fold, 100-fold, or more.

Embodiment 5: The method of embodiment 2 or 3, wherein the activation and/or activity of the PI3K/AKT/mTOR pathway is decreased by at least 10%, 15%, 20%, 25%,30%, 40%, 50%, 60%, 70%, 80%, 90%, 100%, or more.

Embodiment 6: The method of any one of the embodiments 2-5, wherein the anti-HER3 blocker, optionally the anti-HER3 antibody, fragment or equivalent thereof, inhibits PI3K/AKT/mTOR activity.

Embodiment 7: The method of any one of the embodiments 1-6, wherein the cancer expresses a PIK3CA gene comprising a modification.

Embodiment 8: The method of embodiment 7, wherein the modification comprises a substitution in the gene that induced an amino acid mutation.

Embodiment 9: The method of embodiment 8, wherein the amino acid mutation in a PIK3CA protein comprises a mutation at R115, Y343, G363, E542, E545, C971, R975, or H1047, or a combination thereof, wherein the positions correspond to amino acid positions set forth in UniProtKB accession no. P42336.2.

Embodiment 10: The method of any one of the embodiments 7-9, wherein the amino acid mutation comprises R115L, Y343C, G363A, E542K, E545K, E545K, C971R, R975S, H1047L, or H1047R.

Embodiment 11: The method of any one of the embodiments 1-10, wherein the cancer or a cancer cell obtained from the subject’s head and neck cancer expresses a modification in RAS, AKT, PTEN, mTOR, TSC1, TSC2, PIK3CG, PIK3R1, PIK3R5, PIK3AP1, PIK3C2G, or a combination thereof.

Embodiment 12: The method of any one of the embodiments 1-11, wherein the immune checkpoint modulator is a PD-1 inhibitor.

Embodiment 13: The method of embodiment 12, wherein the PD-1 inhibitor comprises one or more of cemiplimab, nivolumab, pembrolizumab, avelumab, durvalumab, or atezolizumab or an equivalent of each thereof.

Embodiment 14: The method of any one of the embodiments 1-13, wherein the head and neck cancer is a human papillomavirus (HPV)-positive cancer.

Embodiment 15: The method of any one of the embodiments 1-14, wherein the head and neck cancer or cancer cell is selected from: laryngeal and hypopharyngeal cancer, nasal cavity and paranasal sinus cancer, nasopharyngeal cancer, oral and oropharyngeal cancer, or salivary gland cancer.

Embodiment 16: The method of any one of the embodiments 1-15, wherein the head and neck cancer or cell comprises or is a metastatic squamous neck cancer with occult primary.

Embodiment 17: The method of any one of the embodiments 1-16, wherein the head and neck cancer or cell comprises or is a metastatic head and neck cancer.

Embodiment 18: The method of any one of the embodiments 1-16, wherein the head and neck cancer is a relapsed or refractory head and neck cancer.

Embodiment 19: The method of any one of the embodiments 1-18, wherein the head and neck cancer is resistant to a PD-1 treatment in the absence of the anti-HER3 blocker, optionally the anti-HER3 antibody, fragment or equivalent thereof.

Embodiment 20: The method of any one of the embodiments 1-19, wherein the anti-HER3 antibody is a monoclonal antibody, fragment or equivalent thereof, optionally selected from CDX-3379 or an equivalent thereof.

Embodiment 21: The method of any one of the embodiments 1-20, wherein the anti-HER3 antibody is a full-length antibody or a fragment thereof.

Embodiment 22: The method of any one of the embodiments 1-21, wherein the anti-HER3 blocker, optionally the anti-HER3 antibody, fragment or equivalent thereof decreases phosphorylated HER3 protein in the subject.

Embodiment 23: The method of any one of the embodiments 1-22, wherein the anti-HER3 blocker, optionally the anti-HER3 antibody, fragment or equivalent thereof impairs or inhibits HER3 phosphorylation.

Embodiment 24: The method of any one of the embodiments 1-23, wherein the anti-HER3 blocker, optionally the anti-HER3 antibody, fragment or equivalent thereof decreases or inhibits ribosomal protein S6 (pS6) phosphorylation.

Embodiment 25: The method of any one of the embodiments 1-24, wherein administration of the anti-HER3 blocker, optionally the anti-HER3 antibody, fragment or equivalent thereof, and the immune checkpoint modulator promotes accumulation of tumor-infiltration lymphocytes.

Embodiment 26: The method of any one of the embodiments 1-25, wherein administration of the anti-HER3 blocker, optionally the anti-HER3 antibody, fragment or equivalent thereof, and the immune checkpoint modulator increases CD8+ T cells in the subject.

Embodiment 27: The method of any one of the embodiments 1-26, wherein the anti-HER3 blocker, optionally the anti-HER3 antibody, fragment or equivalent thereof is formulated for local administration.

Embodiment 28: The method of any one of the embodiments 1-26, wherein the anti-HER3 blocker, optionally the anti-HER3 antibody, fragment or equivalent thereof is formulated for systemic administration.

Embodiment 29: The method of any one of the embodiments 1-28, wherein the immune checkpoint modulator is formulated for local administration.

Embodiment 30: The method of any one of the embodiments 1-28, wherein the immune checkpoint modulator is formulated for systemic administration.

Embodiment 31: The method of any one of the embodiments 1-30, wherein the anti-HER3 blocker, optionally the anti-HER3 antibody, fragment or equivalent thereof, the immune checkpoint modulator, or a combination thereof are formulated for parenteral administration.

Embodiment 32: The method of any one of the embodiments 1-31, wherein the anti-HER3 blocker, optionally the anti-HER3 antibody, fragment or equivalent thereof, the immune checkpoint modulator, or a combination thereof are formulated for intravenous, subcutaneous, intramuscular, intranasal, intra-arterial, intra-articular, intradermal, intraosseous infusion, intraperitoneal, or intratechal administration.

Embodiment 33: The method of embodiment 1, further comprising administering an additional therapeutic agent or an additional therapy to the subject.

Embodiment 34: The method of embodiment 33, wherein the additional therapeutic agent comprises a chemotherapeutic agent, an immunotherapeutic agent, a targeted therapy, radiation therapy, or a combination thereof.

Embodiment 35: The method of embodiment 33 or 34, wherein the additional therapeutic agent comprises a first-line therapy.

Embodiment 36: The method of embodiment 33 or 34, wherein the additional therapeutic agent comprises a second-line therapy, a third-line therapy, a fourth-line therapy, or a fifth-line therapy.

Embodiment 37: The method of embodiment 33, wherein the additional therapy is surgery.

Embodiment 38: The method of any one of the embodiments 1-37, wherein the anti-HER3 blocker, optionally the anti-HER3 antibody, fragment or equivalent thereof, the immune checkpoint modulator, and optionally the additional therapeutic agent are administered simultaneously.

Embodiment 39: The method of any one of the embodiments 1-37, wherein the anti-HER3 blocker, optionally the anti-HER3 antibody, fragment or equivalent thereof, the immune checkpoint modulator, and optionally the additional therapeutic agent are administered sequentially.

Embodiment 40: The method of embodiment 39, wherein the anti-HER3 blocker, optionally the anti-HER3 antibody, fragment or equivalent thereof is administered to the subject prior to administering the immune checkpoint modulator, and optionally the additional therapeutic agent.

Embodiment 41: The method of embodiment 39, wherein the anti-HER3 blocker, optionally the anti-HER3 antibody, fragment or equivalent thereof is administered to the subject after administering the immune checkpoint modulator, and optionally the additional therapeutic agent.

Embodiment 42: The method of any one of the embodiments 1-41, wherein the method enhances one or more of: inhibiting metastasis, progression free survival, tumor growth, or overall survival or decreases toxicity.

Embodiment 43: The method of any one of the embodiments 1-42, wherein the method enhances tumor regression.

Embodiment 44: A method of modulating a head and neck cancer cell proliferation, comprising:
contacting a plurality of cells comprising a head and neck cancer cell and an immune cell with an anti-HER3 blocker, optionally an anti-HER3 antibody, fragment or equivalent thereof, and an immune checkpoint modulator for a time sufficient to induce decreased activity level of the PI3K/AKT/mTOR pathway in the head and neck cancer cell, whereby the decreased activity level of the PI3K/AKT/mTOR pathway increases susceptibility of the head and neck cancer cell to an activated immune cell, thereby impairing or inhibiting proliferation of the head and neck cancer cell.

Embodiment 45: The method of embodiment 44, wherein the immune checkpoint modulator binds to the immune cell to generate the activated immune cell.

Embodiment 46: The method of embodiment 44, wherein the activity level of the PI3K/AKT/mTOR pathway is decreased by at least 1-fold, 2-fold, 3-fold, 4-fold, 5-fold, 6-fold, 7-fold, 8-fold, 9-fold, 10-fold, 20 fold, 30-fold, 50-fold, 100-fold, or more.

Embodiment 47: The method of embodiment 44, wherein the activity level of the PI3K/AKT/mTOR pathway is decreased by at least 10%, 15%, 20%, 25%,30%, 40%, 50%, 60%, 70%, 80%, 90%, 100%, or more.

Embodiment 48: The method of any one of the embodiments 44-47, wherein the immune checkpoint modulator is a PD-1 inhibitor.

Embodiment 49: The method of embodiment 48, wherein the PD-1 inhibitor is cemiplimab, nivolumab, pembrolizumab, avelumab, durvalumab, or atezolizumab.

Embodiment 50: The method of any one of the embodiments 44-49, wherein the head and neck cancer cell expresses a PIK3CA gene comprising a modification.

Embodiment 51: The method of embodiment 50, wherein the modification comprises a substitution in the gene that induced an amino acid mutation.

Embodiment 52: The method of embodiment 50 or 51, wherein the amino acid mutation in a PIK3CA protein comprises a mutation at R115, Y343, G363, E542, E545, C971, R975, or H1047, wherein the positions correspond to amino acid positions set forth in UniProtKB accession no. P42336.2.

Embodiment 53: The method of any one of the embodiments 50-52, wherein the amino acid mutation comprises R115L, Y343C, G363A, E542K, E545K, E545K, C971R, R975S, H1047L, or H1047R.

Embodiment 54: The method of any one of the embodiments 44-53, wherein the plurality of cells are located within a tumor microenvironment.

Embodiment 55: The method of any one of the embodiments 44-54, wherein the method is an in vivo method.

Embodiment 56: The method of any one of the embodiments 44-54, wherein the method is an in vitro method.

Embodiment 57: The method of any one of the embodiments 44-54, wherein the method is an ex vivo method.

Embodiment 58: A method of selecting a subject for treatment with an anti-human epidermal growth factor receptor 3 (HER3) blocker, optional an anti-HER3 antibody, fragment or equivalent thereof, and an immune checkpoint modulator, wherein the subject has a head and neck cancer, the method comprising the steps of:
determining whether the subject can benefit a treatment with the anti-human epidermal growth factor receptor 3 (HER3) antibody, fragment or equivalent thereof, and the immune checkpoint modulator by:
performing or having performed a diagnostic assay on a biological sample isolated from the subject to determine if the subject can benefit the treatment with the anti-human epidermal growth factor receptor 3 (HER3) blocker, optionally the anti-HER3 antibody, fragment or equivalent thereof, and the immune checkpoint modulator; and
if the subject can benefit the treatment, administering to the subject the anti-human epidermal growth factor receptor 3 (HER3) blocker, optionally the anti-HER3 antibody, fragment or equivalent thereof, and the immune checkpoint modulator; or
if the subject cannot benefit the treatment, do not administer the treatment.

Embodiment 59: The method of embodiment 58, wherein the subject can benefit the treatment if the subject expresses a level of PD-1 on a plurality of activated T cells that when blocked, is sufficient to activate tumor-killing activity of the plurality of T cells.

Embodiment 60: The method of embodiment 59, wherein the level of PD-1 expression on the plurality of activated T cells is elevated compared to a level of PD-1 expression on a similar number of T cells obtained from a healthy subject.

Embodiment 61: The method of embodiment 60, wherein the level of PD-1 expression is elevated by about 10%, 20%, 30%, 40%, 50%, 60%, 70%, 80%, 90%, 100%, or more.

Embodiment 62: The method of embodiment 60, wherein the level of PD-1 expression is elevated by about 1-fold, 2-fold, 3-fold, 4-fold, 5-fold, 6-fold, 7-fold, 8-fold, 9-fold, 10-fold, 20-fold, 50-fold, or more.

Embodiment 63: The method of embodiment 58, wherein the subject can benefit the treatment if cells of the head and neck cancer express an elevated level of HER3.

Embodiment 64: The method of embodiment 63, wherein the elevated level of HER3 is compared to an expression level of HER3 in a healthy subject.

Embodiment 65: The method of embodiment 63 or 64, wherein the expression level of HER3 is elevated by about 10%, 20%, 30%, 40%, 50%, 60%, 70%, 80%, 90%, 100%, or more.

Embodiment 66: The method of embodiment 63 or 64, wherein the expression level of HER3 is elevated by about 1-fold, 2-fold, 3-fold, 4-fold, 5-fold, 6-fold, 7-fold, 8-fold, 9-fold, 10-fold, 20-fold, 50-fold, or more.

Embodiment 67: The method of embodiment 58, wherein the subject can benefit the treatment if cells of the head and neck cancer have an elevated activation or activity level in the PI3K/AKT/mTOR pathway.

Embodiment 68: The method of embodiment 67, wherein the elevated activation or activity level in the PI3K/AKT/mTOR pathway is compared to an activation or activity level of the PI3K/AKT/mTOR pathway of cells in a healthy subject.

Embodiment 69: The method of embodiment 67 or 68, wherein the activation or activity level in the PI3K/AKT/mTOR pathway is elevated by about 10%, 20%, 30%, 40%, 50%, 60%, 70%, 80%, 90%, 100%, or more.

Embodiment 70: The method of embodiment 67 or 68, wherein the activation or activity level in the PI3K/AKT/mTOR pathway is elevated by about 1-fold, 2-fold, 3-fold, 4-fold, 5-fold, 6-fold, 7-fold, 8-fold, 9-fold, 10-fold, 20-fold, 50-fold, or more.

Embodiment 71: The method of embodiment 58, wherein the subject can benefit the treatment if cells of the head and neck cancer express a PIK3CA gene comprising a modification that results in an elevated activation or activity level of the PI3K/AKT/mTOR pathway.

Embodiment 72: The method of embodiment 71, wherein the modification comprises a substitution in the gene that induced an amino acid mutation.

Embodiment 73: The method of embodiment 72, wherein the amino acid mutation in a PIK3CA protein comprises a mutation at R115, Y343, G363, E542, E545, C971, R975, or H1047, or a combination thereof, wherein the positions correspond to amino acid positions set forth in UniProtKB accession no. P42336.2.

Embodiment 74: The method of any one of the embodiments 71-73, wherein the amino acid mutation comprises R115L, Y343C, G363A, E542K, E545K, E545K, C971R, R975S, H1047L, or H1047R.

Embodiment 75: The method of any one of the embodiments 58-74, wherein the biological sample is a tumor biopsy sample.

Embodiment 76: The method of any one of the embodiments 58-74, wherein the biological sample is a liquid sample, optionally a blood sample or peripheral blood lymphocytes.

Embodiment 77: The method of any one of the embodiments 58-74, wherein the biological sample is a cell-free DNA sample.

Embodiment 78: The method of any one of the embodiments 58-77, wherein the diagnostic assay is a microarray analysis, quantitative PCR analysis, or RNA sequencing analysis.

Embodiment 79: The method of any one of the embodiments 58-77, wherein the diagnostic assay is an immunofluorescence analysis.

Embodiment 80: The method of any one of the embodiments 58-79, wherein the immune checkpoint modulator is a PD-1 inhibitor.

Embodiment 81: The method of embodiment 80, wherein the PD-1 inhibitor is cemiplimab, nivolumab, pembrolizumab, avelumab, durvalumab, or atezolizumab.

Embodiment 82: The method of any one of the embodiments 58-81, wherein the head and neck cancer is a human papillomavirus (HPV)-positive tumor.

Embodiment 83: The method of any one of the embodiments 58-82, wherein the head and neck cancer is selected from: laryngeal and hypopharyngeal cancer, nasal cavity and paranasal sinus cancer, nasopharyngeal cancer, oral and oropharyngeal cancer, or salivary gland cancer.

Embodiment 84: The method of any one of the embodiments 58-83, wherein the head and neck cancer is a metastatic squamous neck cancer with occult primary.

Embodiment 85: The method of any one of the embodiments 58-84, wherein the head and neck cancer is a metastatic head and neck cancer.

Embodiment 86: The method of any one of the embodiments 58-84, wherein the head and neck cancer is a relapsed or refractory head and neck cancer.

Embodiment 87: The method of any one of the embodiments 58-86, wherein the head and neck cancer is resistant to a PD-1 treatment in the absence of the anti-HER3 blocker, optionally the anti-HER3 antibody, fragment or equivalent thereof.

Embodiment 88: The method of any one of the embodiments 58-87, wherein the anti-HER3 antibody is a monoclonal antibody, fragment or equivalent thereof, optionally selected from CDX-3379.

Embodiment 89: The method of any one of the embodiments 58-88, wherein the anti-HER3 antibody is a full-length antibody or a fragment thereof.

Embodiment 90: The method of any one of the embodiments 58-89, wherein the anti-HER3 blocker, optionally the anti-HER3 antibody, fragment or equivalent thereof is formulated for local administration.

Embodiment 91: The method of any one of the embodiments 58-89, wherein the anti-HER3 blocker, optionally the anti-HER3 antibody, fragment or equivalent thereof is formulated for systemic administration.

Embodiment 92: The method of any one of the embodiments 58-91, wherein the immune checkpoint modulator is formulated for local administration.

Embodiment 93: The method of any one of the embodiments 58-91, wherein the immune checkpoint modulator is formulated for systemic administration.

Embodiment 94: The method of any one of the embodiments 58-93, wherein the anti-HER3 blocker, optionally the anti-HER3 antibody, fragment or equivalent thereof, the immune checkpoint modulator, or a combination thereof are formulated for parenteral administration.

Embodiment 95: The method of any one of the embodiments 58-94, wherein the anti-HER3 blocker, optionally the anti-HER3 antibody, fragment or equivalent thereof, the immune checkpoint modulator, or a combination thereof are formulated for intravenous, subcutaneous, intramuscular, intranasal, intra-arterial, intra-articular, intradermal, intraosseous infusion, intraperitoneal, or intratechal administration.

Embodiment 96: The method of embodiment 58, further comprising administering an additional therapeutic agent or an additional therapy to the subject.

Embodiment 97: The method of embodiment 96, wherein the additional therapeutic agent comprises chemotherapeutic agent, an immunotherapeutic agent, a targeted therapy, radiation therapy, or a combination thereof.

Embodiment 98: The method of embodiment 96 or 97, wherein the additional therapeutic agent comprises a first-line therapy.

Embodiment 99: The method of embodiment 96 or 97, wherein the additional therapeutic agent comprises a second-line therapy, a third-line therapy, a fourth-line therapy, or a fifth-line therapy.

Embodiment 100: The method of embodiment 96, wherein the additional therapy is surgery.

Embodiment 101: The method of any one of the embodiments 58-100, wherein the anti-HER3 blocker, optionally the anti-HER3 antibody, fragment or equivalent thereof, the immune checkpoint modulator, and optionally the additional therapeutic agent are administered simultaneously.

Embodiment 102: The method of any one of the embodiments 58-100, wherein the anti-HER3 blocker, optionally the anti-HER3 antibody, fragment or equivalent thereof, the immune checkpoint modulator, and optionally the additional therapeutic agent are administered sequentially.

Embodiment 103: The method of embodiment 102, wherein the anti-HER3 blocker, optionally the anti-HER3 antibody, fragment or equivalent thereof is administered to the subject prior to administering the immune checkpoint modulator, and optionally the additional therapeutic agent.

Embodiment 104: The method of embodiment 102, wherein the anti-HER3 blocker, optionally the anti-HER3 antibody, fragment or equivalent thereof is administered to the subject after administering the immune checkpoint modulator, and optionally the additional therapeutic agent.

Embodiment 105: The method of any of the preceding embodiments, wherein the subject is a mammal or a human.

Embodiment 106: A pharmaceutical formulation for use in treating a head and neck cancer in a subject in need thereof, comprising:
an anti-human epidermal growth factor receptor 3 (HER3) blocker, optionally an anti-HER3 antibody, fragment or equivalent thereof;
an immune checkpoint modulator; and
a pharmaceutically acceptable carrier.

Embodiment 107: The pharmaceutical formulation of embodiment 106, wherein the immune checkpoint modulator is a PD-1 inhibitor.

Embodiment 108: The pharmaceutical formulation of embodiment 107, wherein the PD-1 inhibitor is cemiplimab, nivolumab, pembrolizumab, avelumab, durvalumab, or atezolizumab.

Embodiment 109: The pharmaceutical formulation of any one of the embodiments 106-108, wherein a cancer cell of the subject’s head and neck cancer expresses a PIK3CA gene comprising a modification.

Embodiment 110: The pharmaceutical formulation of embodiment 109, wherein the modification comprises a substitution in the gene that induced an amino acid mutation.

Embodiment 111: The pharmaceutical formulation of embodiment 110, wherein the amino acid mutation in a PIK3CA protein comprises a mutation at R115, Y343, G363, E542, E545, C971, R975, or H1047, or a combination thereof, wherein the positions correspond to amino acid positions set forth in UniProtKB accession no. P42336.2.

Embodiment 112: The pharmaceutical formulation of any one of the embodiments 109-111, wherein the amino acid mutation comprises R115L, Y343C, G363A, E542K, E545K, E545K, C971R, R975S, H1047L, or H1047R.

Embodiment 113: The pharmaceutical formulation of any one of the embodiments 106-112, wherein a cancer cell obtained from the subject’s head and neck cancer expresses a modification in RAS, AKT, PTEN, mTOR, TSC1, TSC2, PIK3CG, PIK3R1, PIK3R5, PIK3AP1, PIK3C2G, or a combination thereof.

Embodiment 114: The pharmaceutical formulation of any one of the embodiments 106-113, wherein the head and neck cancer is a human papillomavirus (HPV)-positive tumor.

Embodiment 115: The pharmaceutical formulation of any one of the embodiments 106-114, wherein the head and neck cancer is selected from: laryngeal and hypopharyngeal cancer, nasal cavity and paranasal sinus cancer, nasopharyngeal cancer, oral and oropharyngeal cancer, or salivary gland cancer.

Embodiment 116: The pharmaceutical formulation of any one of the embodiments 106-115, wherein the head and neck cancer is a metastatic squamous neck cancer with occult primary.

Embodiment 117: The pharmaceutical formulation of any one of the embodiments 106-116, wherein the head and neck cancer is a metastatic head and neck cancer.

Embodiment 118: The pharmaceutical formulation of any one of the embodiments 106-116, wherein the head and neck cancer is a relapsed or refractory head and neck cancer.

Embodiment 119: The pharmaceutical formulation of any one of the embodiments 106-118, wherein the head and neck cancer is resistant to a PD-1 treatment in the absence of the anti-HER3 antibody, fragment or equivalent thereof.

Embodiment 120: The pharmaceutical formulation of any one of the embodiments 106-119, wherein the anti-HER3 antibody is a monoclonal antibody, fragment or equivalent thereof, optionally selected from CDX-3379.

Embodiment 121: The pharmaceutical formulation of any one of the embodiments 106-120, wherein the anti-HER3 antibody is a full-length antibody or a fragment thereof.

Embodiment 122: The pharmaceutical formulation of any one of the embodiments 106-121, wherein the anti-HER3 blocker, optionally the anti-HER3 antibody, fragment or equivalent thereof, and the immune checkpoint modulator is formulated as a single dosage form.

Embodiment 123: The pharmaceutical formulation of any one of the embodiments 106-121, wherein the anti-HER3 blocker, optionally the anti-HER3 antibody, fragment or equivalent thereof, and the immune checkpoint modulator is formulated as separate dosage forms.

Embodiment 124: The pharmaceutical formulation of any one of the embodiments 106-123, wherein the pharmaceutical formulation further comprises an additional therapeutic agent.

Embodiment 125: The pharmaceutical formulation of embodiment 124, wherein the additional therapeutic agent comprises chemotherapeutic agent, an immunotherapeutic agent, a targeted therapy, radiation therapy, or a combination thereof.

Embodiment 126: The pharmaceutical formulation of embodiment 124 or 125, wherein the additional therapeutic agent comprises a first-line therapy.

Embodiment 127: The pharmaceutical formulation of embodiment 124 or 125, wherein the additional therapeutic agent comprises a second-line therapy, a third-line therapy, a fourth-line therapy, or a fifth-line therapy.

Embodiment 128: A kit comprising a pharmaceutical formulation of embodiments 106-127; optionally including instructions of use.

Other aspects are set forth within the following claims.

### REFERENCES

1 Siegel, R. L., Miller, K. D. & Jemal, A. Cancer statistics, 2019. CA Cancer J Clin 69, 7-34, doi:10.3322/caac.21551 (2019).
2 Siegel, R. L., Miller, K. D. & Jemal, A. Cancer statistics, 2016. CA: a cancer journal for clinicians, doi:10.3322/caac.21332 (2016).
3 Mao, L., Hong,W. K. & Papadimitrakopoulou, V. A. Focus on head and neck cancer. Cancer Cell 5, 311-316 (2004).
4 Chaturvedi, A. K. et al. Human papillomavirus and rising oropharyngeal cancer incidence in the United States. Journal of Clinical Oncology 29, 4294-4301, doi:10.1200/JCO.2011.36.4596 (2011).
5 Chaturvedi, A. K., Engels, E. A., Anderson, W. F. & Gillison, M. L. Incidence trends for human papillomavirus-related and -unrelated oral squamous cell carcinomas in the United States. Journal of Clinical Oncology 26, 612-619, doi:10.1200/JCO.2007.14.1713 (2008).
6 Cohen, E. E. et al. American Cancer Society Head and Neck Cancer Survivorship Care Guideline. CA Cancer J Clin 66, 203-239, doi:10.3322/caac.21343 (2016).
7 Pulte, D. & Brenner, H. Changes in survival in head and neck cancers in the late 20th and early 21st century: a period analysis. Oncologist 15, 994-1001, doi:10.1634/theoncologist.2009-0289 (2010).
8 Miller, K. D. et al. Cancer treatment and survivorship statistics, 2016. CA: a cancer journal for clinicians, doi:10.3322/caac.21349 (2016).
9 Kaneda, M. M. et al. PI3Kgamma is a molecular switch that controls immune suppression. Nature 539, 437-442, doi:10.1038/nature19834 (2016).
10 Ferris, R. L. Immunology and Immunotherapy of Head and Neck Cancer. J Clin Oncol 33, 3293-3304, doi:10.1200/JCO.2015.61.1509 (2015).
11 Duray, A., Demoulin, S., Hubert, P., Delvenne, P. & Saussez, S. Immune suppression in head and neck cancers: a review. Clin Dev Immunol 2010, 701657, doi:10.1155/2010/701657 (2010).
12 Lyford-Pike, S. et al. Evidence for a role of the PD-1:PD-L1 pathway in immune resistance of HPV-associated head and neck squamous cell carcinoma. Cancer Res 73, 1733-1741, doi:10.1158/0008-5472.CAN-12-2384 (2013).
13 Badoual, C. et al. PD-1-expressing tumor-infiltrating T cells are a favorable prognostic biomarker in HPV-associated head and neck cancer. Cancer Res 73, 128-138, doi:10.1158/0008-5472.CAN-12-2606 (2013).
14 Ukpo, O. C., Thorstad, W. L. & Lewis, J. S., Jr. B7-H1 expression model for immune evasion in human papillomavirus-related oropharyngeal squamous cell carcinoma. Head Neck Pathol 7, 113-121, doi:10.1007/s12105-012-0406-z (2013).
15 Ferris, R. L. et al. Nivolumab for Recurrent Squamous-Cell Carcinoma of the Head and Neck. N Engl J Med 375, 1856-1867, doi:10.1056/NEJMoa1602252 (2016).
16 Chow, L. Q. M. et al. Antitumor activity of pembrolizumab in biomarker-unselected patients with recurrent and/or metastatic head and neck squamous cell carcinoma: Results from the phase Ib KEYNOTE-012 expansion cohort. Journal of Clinical Oncology 34, 3838-3845, doi:10.1200/JCO.2016.68.1478 (2016).
17 Cohen, E. E. W. et al. Pembrolizumab versus methotrexate, docetaxel, or cetuximab for recurrent or metastatic head-and-neck squamous cell carcinoma (KEYNOTE-040): a randomised, open-label, phase 3 study. Lancet 393, 156-167, doi:10.1016/S0140-6736(18)31999-8 (2019).
18 Stevanović, S. et al. Complete regression of metastatic cervical cancer after treatment with human papillomavirus-targeted tumor-infiltrating T cells. Journal of Clinical Oncology 33, 1543-1550, doi:10.1200/JCO.2014.58.9093 (2015).
19 Lui, V. W. et al. Frequent mutation of the PI3K pathway in head and neck cancer defines predictive biomarkers. Cancer discovery 3, 761-769 (2013).
20 Pickering, C. R. et al. Integrative genomic characterization of oral squamous cell carcinoma identifies frequent somatic drivers. Cancer discovery 3, 770-781 (2013).
21 Iglesias-Bartolome, R., Martin, D. & Gutkind, J. S. Exploiting the head and neck cancer oncogenome: widespread PI3K-mTOR pathway alterations and novel molecular targets. Cancer discovery 3, 722-725, doi:10.1158/2159-8290.CD-13-0239 (2013).
22 Cancer Genome Atlas, N. Comprehensive genomic characterization of head and neck squamous cell carcinomas. Nature 517, 576-582, doi:10.1038/nature14129 (2015).
23 Network, C. G. A. Comprehensive genomic characterization of head and neck squamous cell carcinomas. Nature 517, 576-582 (2015).
24 Amornphimoltham, P. et al. Persistent activation of the Akt pathway in head and neck squamous cell carcinoma: a potential target for UCN-01. Clinical Cancer Research 10, 4029-4037, doi:10.1158/1078-0432.ccr-03-0249 (2004).
25 Molinolo, A. A. et al. Dissecting the Akt/mammalian target of rapamycin signaling network: emerging results from the head and neck cancer tissue array initiative. Clin Cancer Res 13, 4964-4973, doi:10.1158/1078-0432.CCR-07-1041 (2007).
26 Amornphimoltham, P. et al. Mammalian target of rapamycin, a molecular target in squamous cell carcinomas of the head and neck. Cancer Res 65, 9953-9961, doi:10.1158/0008-5472.CAN-05-0921 (2005).
27 Molinolo, A. A. et al. mTOR as a molecular target in HPV-associated oral and cervical squamous carcinomas. Clinical Cancer Research 18, 2558-2568, doi:10.1158/1078-0432.CCR-11-2824 (2012).
28 Raimondi, A. R., Molinolo, A. & Gutkind, J. S. Rapamycin prevents early onset of tumorigenesis in an oral-specific K-ras and p53 two-hit carcinogenesis model. Cancer Res 69, 4159-4166, doi:10.1158/0008-5472.can-08-4645 (2009).
29 Czerninski, R., Amornphimoltham, P., Patel, V., Molinolo, A. A. & Gutkind, J. S. Targeting mammalian target of rapamycin by rapamycin prevents tumor progression in an oral-specific chemical carcinogenesis model. Cancer Prev Res (Phila) 2, 27-36, doi:10.1158/1940-6207.CAPR-08-0147 (2009).
30 Coppock, J. D. et al. Improved clearance during treatment of HPV-positive head and neck cancer through mTOR inhibition. Neoplasia 15, 620-630 (2013).
31 Patel, V. et al. Decreased Lymphangiogenesis and Lymph Node Metastasis by mTOR Inhibition in Head and Neck Cancer. Cancer research, doi:10.1158/0008-5472.CAN-10-3192 (2011).
32 Saito, K. et al. Longitudinal imaging studies of tumor microenvironment in mice treated with the mTOR inhibitor rapamycin. PLoS One 7, e49456, doi:10.1371/journal.pone.0049456 (2012).
33 Squarize, C. H., Castilho, R. M. & Gutkind, J. S. Chemoprevention and treatment of experimental Cowden’s disease by mTOR inhibition with rapamycin. Cancer Res 68, 7066-7072, doi:10.1158/0008-5472.CAN-08-0922 (2008).
34 Sun, Z.-J. et al. Chemopreventive and chemotherapeutic actions of mTOR inhibitor in genetically defined head and neck squamous cell carcinoma mouse model. Clinical Cancer Research 18, 5304-5313 (2012).
35 Day, T. A. et al. Inhibition of mTOR Signaling and Clinical Activity of Rapamycin in Head and Neck Cancer in a Window of Opportunity Trial. Clin Cancer Res, doi:10.1158/1078-0432.CCR-18-2024 (2018).
36 Falchook, G. S. et al. Safety, pharmacokinetics (PK), pharmacodynamics (Pd), and antitumor activity in a phase 1b study evaluating anti-ErbB3 antibody KTN3379 in adults with advanced tumors alone and with targeted therapies. Journal of Clinical Oncology 34, 2501-2501, doi:10.1200/JCO.2016.34.15_suppl.2501 (2016).
37 Bauman, J. E. et al. A phase 2, multicenter, open-label study to evaluate the efficacy and safety of CDX-3379 in combination with cetuximab in patients with advanced head and neck squamous cell carcinoma (HNSCC). Journal of Clinical Oncology 36, TPS6091-TPS6091, doi:10.1200/JCO.2018.36.15_suppl.TPS6091 (2018).
38 Brand, T. M. et al. Cross-talk Signaling between HER3 and HPV16 E6 and E7 Mediates Resistance to PI3K Inhibitors in Head and Neck Cancer. Cancer Res 78, 2383-2395, doi:10.1158/0008-5472.CAN-17-1672 (2018).
39 Alvarado, D. et al. ErbB activation signatures as potential biomarkers for anti-ErbB3 treatment in HNSCC. PLoS One 12, e0181356, doi:10.1371/journal.pone.0181356 (2017).
40 Brand, T. M. et al. Human Papillomavirus Regulates HER3 Expression in Head and Neck Cancer: Implications for Targeted HER3 Therapy in HPV(+) Patients. Clin Cancer Res 23, 3072-3083, doi:10.1158/1078-0432.CCR-16-2203 (2017).
41 Wheeler, D. L. et al. Mechanisms of acquired resistance to cetuximab: role of HER (ErbB) family members. Oncogene 27, 3944-3956, doi:10.1038/onc.2008.19 (2008).
42 Iida, M. et al. Targeting the HER Family with Pan-HER Effectively Overcomes Resistance to Cetuximab. Mol Cancer Ther 15, 2175-2186, doi:10.1158/1535-7163.MCT-16-0012 (2016).
43 Vlacich, G. & Coffey, R. J. Resistance to EGFR-targeted therapy: a family affair. Cancer Cell 20, 423-425, doi:10.1016/j.ccr.2011.10.006 (2011).
44 Wang, D. et al. HER3 Targeting Sensitizes HNSCC to Cetuximab by Reducing HER3 Activity and HER2/HER3 Dimerization: Evidence from Cell Line and Patient-Derived Xenograft Models. Clin Cancer Res 23, 677-686, doi:10.1158/1078-0432.CCR-16-0558 (2017).
45 Lemmon, M. A. & Schlessinger, J. Cell signaling by receptor tyrosine kinases. Cell 141, 1117-1134, doi:10.1016/j.cell.2010.06.011 (2010).
46 Grandis, J. R. et al. Levels of TGF-alpha and EGFR protein in head and neck squamous cell carcinoma and patient survival. Journal of the National Cancer Institute 90, 824-832, doi:10.1093/jnci/90.11.824 (1998).
47 Shames, D. S. et al. High heregulin expression is associated with activated HER3 and may define an actionable biomarker in patients with squamous cell carcinomas of the head and neck. PLoS One 8, e56765, doi:10.1371/journal.pone.0056765 (2013).
48 Soltoff, S. P., Carraway, K. L., 3rd, Prigent, S. A., Gullick, W. G. & Cantley, L. C. ErbB3 is involved in activation of phosphatidylinositol 3-kinase by epidermal growth factor. Mol Cell Biol 14, 3550-3558 (1994).
49 Hellyer, N. J., Kim, M. S. & Koland, J. G. Heregulin-dependent activation of phosphoinositide 3-kinase and Akt via the ErbB2/ErbB3 co-receptor. J Biol Chem 276, 42153-42161, doi:10.1074/jbc.M102079200 (2001).
50 Schulze, W. X., Deng, L. & Mann, M. Phosphotyrosine interactome of the ErbB-receptor kinase family. Mol Syst Biol 1, 2005 0008, doi:10.1038/msb4100012 (2005).
51 Jiang, N. et al. Combination of anti-HER3 antibody MM-121/SAR256212 and cetuximab inhibits tumor growth in preclinical models of head and neck squamous cell carcinoma. Mol Cancer Ther 13, 1826-1836, doi:10.1158/1535-7163.MCT-13-1093 (2014).
52 Fayette, J. et al. Randomized Phase II Study of Duligotuzumab (MEHD7945A) vs. Cetuximab in Squamous Cell Carcinoma of the Head and Neck (MEHGAN Study). Front Oncol 6, 232, doi:10.3389/fonc.2016.00232 (2016).
53 Dillon, M. T. et al. Patritumab with Cetuximab plus Platinum-Containing Therapy in Recurrent or Metastatic Squamous Cell Carcinoma of the Head and Neck: An Open-Label, Phase Ib Study. Clin Cancer Res 25, 487-495, doi:10.1158/1078-0432.CCR-18-1539 (2019).
54 Yoshiba, S., Jansen, M., Matsushima, N., Chen, S. & Mendell, J. Population pharmacokinetic analysis of patritumab, a HER3 inhibitor, in subjects with advanced non-small cell lung cancer (NSCLC) or solid tumors. Cancer Chemother Pharmacol 77, 987-996, doi:10.1007/s00280-016-3011-0 (2016).
55 Lee, S. et al. Inhibition of ErbB3 by a monoclonal antibody that locks the extracellular domain in an inactive configuration. Proceedings of the National Academy of Sciences of the United States of America 112, 13225-13230, doi:10.1073/pnas.1518361112 (2015).
56 Xiao, Z. et al. A Potent HER3 Monoclonal Antibody That Blocks Both Ligand-Dependent and -Independent Activities: Differential Impacts of PTEN Status on Tumor Response. Mol Cancer Ther 15, 689-701, doi:10.1158/1535-7163.MCT-15-0555 (2016).
57 Williams, R. et al. Preclinical models of HPV+ and HPV- HNSCC in mice: an immune clearance of HPV+ HNSCC. Head Neck 31, 911-918, doi:10.1002/hed.21040 (2009).
58 Chalivendra, V. et al. Genomic analysis to define molecular basis of aggressiveness in a mouse model of oral cancer. Genom Data 3, 61-62, doi:10.1016/j.gdata.2014.11.006 (2015).
59 Vitale-Cross, L. et al. Chemical carcinogenesis models for evaluating molecular-targeted prevention and treatment of oral cancer. Cancer Prev Res (Phila) 2, 419-422, doi:10.1158/1940-6207.CAPR-09-0058 (2009).
60 Vitale-Cross, L. et al. Metformin prevents the development of oral squamous cell carcinomas from carcinogen-induced premalignant lesions. Cancer Prevention Research 5, 562-573, doi:10.1158/1940-6207.CAPR-11-0502 (2012).
61 Squarize, C. H. et al. PTEN deficiency contributes to the development and progression of head and neck cancer. Neoplasia 15, 461-471 (2013).
62 Sheu, J. J. et al. Functional genomic analysis identified epidermal growth factor receptor activation as the most common genetic event in oral squamous cell carcinoma. Cancer Res 69, 2568-2576, doi:10.1158/0008-5472.CAN-08-3199 (2009).
63 Li, J., Liang, F., Yu, D., Qing, H. & Yang, Y. Development of a 4-nitroquinoline-1-oxide model of lymph node metastasis in oral squamous cell carcinoma. Oral Oncol 49, 299-305, doi:10.1016/j.oraloncology.2012.10.013 (2013).
64 Zhong, R. et al. Notch1 Activation or Loss Promotes HPV-Induced Oral Tumorigenesis. Cancer Res 75, 3958-3969, doi:10.1158/0008-5472.CAN-15-0199 (2015).
65 Tang, X. H. et al. Gene expression profiling signatures for the diagnosis and prevention of oral cavity carcinogenesis-genome-wide analysis using RNA-seq technology. Oncotarget 6, 24424-24435, doi:10.18632/oncotarget.4420 (2015).
66 The Cancer Genome Atlas, N. Comprehensive genomic characterization of head and neck squamous cell carcinomas. Nature 517, 576-582, doi:10.1038/nature14129 http://www.nature.com/nature/journal/v517/n7536/abs/nature14129.html#supplementary-information (2015).
67 Alexandrov, L. B. et al. Mutational signatures associated with tobacco smoking in human cancer. Science 354, 618-622, doi:10.1126/science.aag0299 (2016).

1. A method of treating a head and neck cancer in a subject in need thereof, comprising administering to the subject an anti-human epidermal growth factor receptor 3 (HER3) antibody or a fragment thereof, and an immune checkpoint modulator, wherein:
the anti-HER3 antibody or a fragment thereof modulates, decreases, or inhibits activation and/or activity of the PI3K/AKT/mTOR pathway; and wherein:
a cancer cell obtained from the subject’s head and neck cancer expresses a PIK3CA gene comprising a modification, optionally comprising a mutation selected from R115, Y343, G363, E542, E545, C971, R975, or H1047, or a combination thereof, wherein the positions correspond to amino acid positions set forth in UniProtKB accession no. P42336.2.

2. (canceled)

3. (canceled)

4. (canceled)

5. The method of claim 1, wherein the head and neck cancer is a human papillomavirus (HPV)-positive tumor.

6. The method of claim 1, wherein the head and neck cancer is selected from:
laryngeal and hypopharyngeal cancer, nasal cavity and paranasal sinus cancer, nasopharyngeal cancer, oral and oropharyngeal cancer, or salivary gland cancer;
a metastatic squamous neck cancer with occult primary;
a metastatic head and neck cancer; or
a relapsed or refractory head and neck cancer.

7. The method of claim 1, wherein the head and neck cancer is resistant to a PD-1 treatment in the absence of the anti-HER3 antibody or a fragment thereof.

8. The method of claim 1, wherein the anti-HER3 antibody or a fragment thereof impairs or inhibits HER3 phosphorylation, decreases or inhibits ribosomal protein S6 (pS6) phosphorylation, or a combination thereof.

9. The method of claim 1, wherein the anti-HER3 antibody is CDX-3379.

10. (canceled)

11. (canceled)

12. The method of claim 1, further comprising administering an additional therapeutic agent or an additional therapy to the subject, optionally selected from a chemotherapeutic agent, an immunotherapeutic agent, a targeted therapy, radiation therapy, or a combination thereof.

13. A method of modulating a head and neck cancer cell proliferation, comprising:
contacting a plurality of cells comprising a head and neck cancer cell and an immune cell with an anti-HER3 antibody or a fragment thereof, and an immune checkpoint modulator for a time sufficient to induce decreased activity level of the PI3K/AKT/mTOR pathway in the head and neck cancer cell, whereby the decreased activity level of the PI3K/AKT/mTOR pathway increases susceptibility of the head and neck cancer cell to an activated immune cell, thereby impairing or inhibiting proliferation of the head and neck cancer cell.

14. The method of claim 13, wherein the immune checkpoint modulator is a PD-1 inhibitor, optionally selected from cemiplimab, nivolumab, pembrolizumab, avelumab, durvalumab, and atezolizumab.

15. The method of claim 13, wherein the head and neck cancer cell expresses a PIK3CA gene comprising a modification, optionally comprising a mutation selected from R115, Y343, G363, E542, E545, C971, R975, or H1047, or a combination thereof, wherein the positions correspond to amino acid positions set forth in UniProtKB accession no. P42336.2.

16. The method of claim 13, wherein the plurality of cells are located within a tumor microenvironment.

17. The method of claim 13, wherein the method is an in vivo method.

18. The method of claim 13, wherein the method is an in vitro or an ex vivo method.

19. A method of treating a subject with an anti-human epidermal growth factor receptor 3 (HER3) antibody, fragment or equivalent thereof, and an immune checkpoint modulator, wherein the subject has a head and neck cancer, and wherein the subject can benefit the treatment if the subject expresses a level of PD-1 on a plurality of activated T cells that when blocked, is sufficient to activate tumor-killing activity of the plurality of T cells, the method comprising the steps of:
determining whether the subject can benefit a treatment with the anti-human epidermal growth factor receptor 3 (HER3) antibody or a fragment thereof, and the immune checkpoint modulator by:
performing or having performed a diagnostic assay on a biological sample isolated from the subject to determine if the subject can benefit the treatment with the anti-human epidermal growth factor receptor 3 (HER3) antibody or a fragment thereof, and the immune checkpoint modulator; and
if the subject can benefit the treatment, administering to the subject the anti-human epidermal growth factor receptor 3 (HER3) antibody or a fragment thereof, and the immune checkpoint modulator; or
if the subject cannot benefit the treatment, do not administer the treatment.

20. (canceled)

21. The method of claim 19, wherein the level of PD-1 expression on the plurality of activated T cells is elevated compared to a level of PD-1 expression on a similar number of T cells obtained from a healthy subject.

22. The method of claim 19, wherein the subject can benefit the treatment if cells of the head and neck cancer express an elevated level of HER3, optionally compared to an expression level of HER3 in a healthy subject.

23. The method of claim 19, wherein the subject can benefit the treatment if cells of the head and neck cancer have an elevated activation or activity level in the PI3K/AKT/mTOR pathway.

24. The method of claim 23, wherein the elevated activation or activity level in the PI3K/AKT/mTOR pathway is compared to an activation or activity level of the PI3K/AKT/mTOR pathway of cells in a healthy subject.

25. The method of claim 19, wherein the biological sample is a tumor biopsy sample, a cell-free DNA sample, or a liquid sample, optionally a blood sample or peripheral blood lymphocytes.

26. A pharmaceutical formulation for use in treating a head and neck cancer in a subject in need thereof, comprising:
an anti-human epidermal growth factor receptor 3 (HER3) antibody or a fragment thereof;
an immune checkpoint modulator; and
a pharmaceutically acceptable carrier.

27. (canceled)

28. (canceled)

29. (canceled)

30. (canceled)
